(19) United States
(12) Patent Application Publication
(10) Pub. No.: US 2023/0310455 A1
(43) Pub. Date: Oct. 5, 2023

(54) COMBINATION THERAPIES FOR TREATMENT OF HER2 CANCER

(71) Applicants: Genentech, Inc., South San Francisco, CA (US); Hoffmann-La Roche Inc., Little Falls, NJ (US)

(72) Inventors: Jennifer Lee Schutzman, Belmont, CA (US); Chunyan Song, Redwood City, CA (US); Kyung Wha Song, Union City, CA (US); Eirini Thanopoulou, St Albans (GB); Simon Peter Warburton, Northants (GB); Anwesha Dey, Cupertino, CA (US); Stephanie Royer Joo, San Diego, CA (US)

(73) Assignees: Genentech, Inc., South San Francisco, CA (US); Hoffmann-La Roche Inc., Little Falls, NJ (US)

(21) Appl. No.: 18/208,024

(22) Filed: Jun. 9, 2023

Related U.S. Application Data
(63) Continuation of application No. PCT/US2021/062101, filed on Dec. 7, 2021.
(60) Provisional application No. 63/124,495, filed on Dec. 11, 2020.
(60) Provisional application No. 63/161,153, filed on Mar. 15, 2021.
(60) Provisional application No. 63/209,302, filed on Jun. 10, 2021.

(51) Int. Cl. A61K 31/553; C07K 16/32; A61K 31/337; A61P 35/04; A61K 9/00; A61K 31/565; A61K 31/4196

(52) U.S. Cl. CPC A61K 31/553; C07K 16/32; A61K 31/337; A61P 35/04; A61K 9/0053; A61K 31/565; A61K 31/4196

(57) ABSTRACT

Provided are combination therapies comprising inavolisib (a.k.a. GDC-0077) and other HER2-targeted therapies (e.g., pertuzumab and trastuzumab) for the treatment of HER2-positive cancers; and methods of treating HER2 positive (HER2+) cancers in a patient (preferably a patient with a PIK3CA mutant breast cancer) comprising administering a therapeutically effective amount of inavolisib and a HER2-targeted therapy (e.g., trastuzumab, pertuzumab, or a combination of trastuzumab and pertuzumab).

### CROSS-REFERENCE TO RELATED APPLICATIONS

This application claims the benefit of U.S. Provisional Patent Application No. 63/124,495, filed Dec. 11, 2020; U.S. Provisional Patent Application No. 63/161,153, filed Mar. 15, 2021; and U.S. Provisional Patent Application No. 63/209,302, filed Jun. 10, 2021; the contents of which are incorporated by reference herein in their entireties.

### FIELD OF THE INVENTION

The invention relates generally to treatment of locally advanced or metastatic, PIK3CA-mutated solid tumors, including HER2-positive breast cancer, in patients by administering a PI3K inhibitor, inavolisib (a.k.a. GDC-0077), in combination with other HER2-targeted therapies, for example, pertuzumab and trastuzumab.

### BACKGROUND

Globally, breast cancer is the second most common invasive malignancy and the most common cause of cancer-related mortality in women, with a 5-year survival rate following metastatic diagnosis of approximately 15%.

Phosphatidylinositol 3-kinase (PI3K) is a lipid kinase that upon activation by growth factor receptors and integrins regulates cell proliferation, survival, and migration. PI3K catalyzes the phosphorylation of phosphatidylinositol-4,5-bisphosphate (PIP₂) to generate phosphatidylinositol-3,4,5-triphosphate (PIPS), a second messenger involved in the phosphorylation of AKT and other components in the AKT/mTOR pathway. Up to 70% of breast cancers have some form of molecular aberration of the PI3K/AKT/mTOR pathway. Activating mutations in PIK3CA, encoding the p110α subunit of PI3K, are highly prevalent in breast cancer and solid tumor malignancies.

While inhibitors of PI3Kα have been approved or in clinical development for the treatment of patients with hormone receptor (HR)-positive, HER2-negative, locally advanced or metastatic breast cancer with a PIK3CA mutation, there remains a need for active agents for treatment of HER2-positive cancers.

### SUMMARY OF THE INVENTION

The present disclosure provides a combination therapy comprising inavolisib (GDC-0077) and a HER2-targeted therapy (e.g., trastuzumab, pertuzumab, or a combination of trastuzumab and pertuzumab) for the treatment of HER2-overexpressing breast cancer.

One aspect of the present disclosure provides a combination therapy comprising inavolisib, trastuzumab and pertuzumab for the treatment of PIK3CA-mutated, HER2-positive (HER2+) breast cancer.

The disclosure further provides methods of treating locally advanced or metastatic PIK3CA-mutated (or PIK3CA mutant) HER2-positive breast cancer comprising administering to a patient in need thereof a therapeutically effective amount of inavolisib, or a pharmaceutically acceptable salt thereof, and a HER2-targeted therapy (e.g., trastuzumab, pertuzumab, or a combination of trastuzumab and pertuzumab).

In one aspect, the present disclosure provides a method of treating locally advanced or metastatic PIK3CA-mutated HER2-positive breast cancer comprising administering to a patient in need thereof a therapeutically effective amount of inavolisib, or a pharmaceutically acceptable salt thereof, trastuzumab and pertuzumab.

In one aspect, the present disclosure provides a method of treating locally advanced or metastatic PIK3CA-mutated HER2-positive breast cancer comprising administering to a patient in need thereof a therapeutically effective amount of inavolisib, or a pharmaceutically acceptable salt thereof, trastuzumab, pertuzumab and paclitaxel.

In some embodiments, the patient has locally advanced or metastatic PIK3CA-mutant HER2+breast cancer. In some embodiments, the patient has left ventricular ejection fraction (LVEF) 50% or greater. In some embodiments, the patient is female.

In some embodiments, the patient has hormone receptor positive (HR+) locally advanced or metastatic PIK3CA-mutated HER2-positive breast cancer, and the method further comprises administering to the patient an endocrine therapy (e.g., fulvestrant or letrozole).

Also provided is a combination for use in treating locally advanced or metastatic PIK3CA-mutated (or PIK3CA mutant) HER2-positive breast cancer wherein said combination comprises inavolisib, or a pharmaceutically acceptable salt thereof, and a HER2-targeted therapy (e.g., trastuzumab, pertuzumab, or a combination of trastuzumab and pertuzumab).

Also provided is a use of a combination in the manufacture of a medicament for treating locally advanced or metastatic PIK3CA-mutated (or PIK3CA mutant) HER2-positive breast cancer wherein said combination comprises inavolisib, or a pharmaceutically acceptable salt thereof, and a HER2-targeted therapy (e.g., trastuzumab, pertuzumab, or a combination of trastuzumab and pertuzumab).

In some embodiments, inavolisib (GDC-0077) is adminstered at a 3, 6, or 9 mg daily dose. In one embodiment, inavolisib (GDC-0077) is adminstered orally at a 9 mg daily dose.

In some embodiments, provided is a method of treating HER2-positive breast cancer in a patient having locally advanced or metastatic PIK3CA-mutated HER2-positive breast cancer, the method comprising administering to the patient a combination therapy comprising: inavolisib, trastuzumab and pertuzumab; wherein said combination therapy is administered over a 21-day cycle.

In some embodiments, provided is a method of treating HER2-positive breast cancer in a patient having locally advanced or metastatic PIK3CA-mutated HER2-positive breast cancer, the method comprising administering to the patient a combination therapy comprising a dosing regimen comprising:
a. administering inavolisib QD on days 1-21 of a first 21-day cycle;
b. administering trastuzumab on day 1 of a first 21-day cycle; and
c. administering pertuzumab on day 1 of a first 21-day cycle.

In some embodiments, provided is a method of treating HER2-positive breast cancer in a patient having locally advanced or metastatic PIK3CA-mutated HER2-positive breast cancer, the method comprising administering to the patient a combination therapy comprising a dosing regimen comprising:
a. administering paclitaxel on days 1, 8 and 15 of a first 21-day cycle;
b. administering inavolisib QD on days 2-21 of a first 21-day cycle;
c. administering trastuzumab on day 2 of a first 21-day cycle;
d. administering pertuzumab on day 2 of a first 21-day cycle.
   In some embodiments, the dosing regimen further comprises up to five additional 21-day cycles comprising:
e. administering paclitaxel on days 1, 8 and 15 of each additional 21-day cycle;
f. administering inavolisib on days 1-21 of each additional 21-day cycle;
g. administering trastuzumab on day 1 of each additional 21-day cycle; and
h. administering pertuzumab on day 1 of each additional 21-day cycle.

In some of these embodiments, the method further comprises one or more additional 21-day cycles comprising:
a. administering inavolisib on days 1-21 of each additional 21-day cycle;
b. administering trastuzumab on day 1 of each additional 21-day cycle; and
c. administering pertuzumab on day 1 of each additional 21-day cycle.

Also provided is a combination for use in treating HER2-positive breast cancer in a patient having locally advanced or metastatic PIK3CA-mutated (or PIK3CA mutant) HER2-positive breast cancer, wherein said combination comprises inavolisib, trastuzumab, pertuzumab, and optionally paclitaxel; wherein said combination therapy is administered over a 21-day cycle.

Also provided is a combination for use in treating HER2-positive breast cancer in a patient having locally advanced or metastatic PIK3CA-mutated HER2-positive breast cancer, wherein said combination is administered in a combination therapy comprising a dosing regimen comprising:
a. administering inavolisib QD on days 1-21 of a first 21-day cycle;
b. administering trastuzumab on day 1 of a first 21-day cycle; and
c. administering pertuzumab on day 1 of a first 21-day cycle.

Also provided is a combination for use in treating HER2-positive breast cancer in a patient having locally advanced or metastatic PIK3CA-mutated HER2-positive breast cancer, wherein said combination is administered in a combination therapy comprising a dosing regimen comprising:
a. administering paclitaxel on days 1, 8 and 15 of a first 21-day cycle;
b. administering inavolisib QD on days 2-21 of a first 21-day cycle;
c. administering trastuzumab on day 2 of a first 21-day cycle;
d. administering pertuzumab on day 2 of a first 21-day cycle.
   In some embodiments, the dosing regimen further comprises up to five additional 21-day cycles comprising:
e. administering paclitaxel on days 1, 8 and 15 of each additional 21-day cycle;
f. administering inavolisib on days 1-21 of each additional 21-day cycle;
g. administering trastuzumab on day 1 of each additional 21-day cycle; and
h. administering pertuzumab on day 1 of each additional 21-day cycle.

Also provided is a use of a combination in the manufacture of a medicament for treating HER2-positive breast cancer in a patient having locally advanced or metastatic PIK3CA-mutated (or PIK3CA mutant) HER2-positive breast cancer, wherein said combination comprises inavolisib, trastuzumab, pertuzumab, and optionally paclitaxel; wherein said combination therapy is administered over a 21-day cycle.

Also provided is a use of a combination in the manufacture of a medicament for treating HER2-positive breast cancer in a patient having locally advanced or metastatic PIK3CA-mutated HER2-positive breast cancer, wherein said combination is administered in a combination therapy comprising a dosing regimen comprising:
a. administering inavolisib QD on days 1-21 of a first 21-day cycle;
b. administering trastuzumab on day 1 of a first 21-day cycle; and
c. administering pertuzumab on day 1 of a first 21-day cycle.

Also provided is a use of a combination in the manufacture of a medicament for treating HER2-positive breast cancer in a patient having locally advanced or metastatic PIK3CA-mutated HER2-positive breast cancer, wherein said combination is administered in a combination therapy comprising a dosing regimen comprising:
a. administering paclitaxel on days 1, 8 and 15 of a first 21-day cycle;
b. administering inavolisib QD on days 2-21 of a first 21-day cycle;
c. administering trastuzumab on day 2 of a first 21-day cycle;
d. administering pertuzumab on day 2 of a first 21-day cycle.
   In some embodiments, the dosing regimen further comprises up to five additional 21-day cycles comprising:
e. administering paclitaxel on days 1, 8 and 15 of each additional 21-day cycle;
f. administering inavolisib on days 1-21 of each additional 21-day cycle;
g. administering trastuzumab on day 1 of each additional 21-day cycle; and
h. administering pertuzumab on day 1 of each additional 21-day cycle.

In some of these embodiments, the dosing regimen further comprises one or more additional 21-day cycles comprising:
a. administering inavolisib on days 1-21 of each additional 21-day cycle;
b. administering trastuzumab on day 1 of each additional 21-day cycle; and
c. administering pertuzumab on day 1 of each additional 21-day cycle.

In some of these embodiments, inavolisib is administered at an amount of 9 mg, e.g., in an oral tablet. In some embodiments, trastuzumab is administered at a loading dose of 8 mg/kg for the first 21-day cycle and a dose of 6 mg/kg for each additional 21-day cycle by intravenous (IV) infusion. In some embodiments, pertuzumab is administered at a loading dose of 840 mg for the first 21-day cycle and a dose of 420 mg for each additional 21-day cycle by IV infusion. In some embodiments, paclitaxel is administered at a weekly dose of 80 mg/m² for the first 21-day cycle and up to five additional 21-day cycles by IV infusion.

In some embodiments, the patient has hormone receptor positive (HR+) locally advanced or metastatic PIK3CA-mutated HER2-positive breast cancer. In some of these embodiments, the method further comprises administering to the patient fulvestrant (e.g., once approximately every four weeks at a dose of 500 mg by intramuscular infusion) or letrozole (e.g., daily at a dose of 2.5 mg in an oral tablet).

In a further aspect, provided is a method of inhibiting tumor growth or producing/increasing tumor regression in a patient having locally advanced or metastatic PIK3CA-mutated (or PIK3CA mutant) HER2-positive breast cancer, the method comprising administering to the patient a combination therapy according to the methods detailed herein.

In another aspect, provided is a combination for use in inhibiting tumor growth or producing/increasing tumor regression in a patient having locally advanced or metastatic PIK3CA-mutated HER2-positive breast cancer, according to the combination for use detailed herein.

In another aspect, provided is a use of a combination in the manufacture of a medicament for inhibiting tumor growth or producing/increasing tumor regression in a patient having locally advanced or metastatic PIK3CA-mutated HER2-positive breast cancer, according to the combinations or uses detailed herein.

Further provided is a method of preventing or delaying development of resistance of a tumor (e.g., breast cancer) to a therapy containing a HER2-targeted therapy, said method comprising administering a combination therapy comprising inavolisib, trastuzumab and pertuzumab. In some embodiments, the combination therapy is administered according to any methods as detailed herein.

Also provided is a combination for use in preventing or delaying development of resistance of a tumor (e.g., breast cancer) to a therapy containing a HER2-targeted therapy, wherein said combination comprises inavolisib, trastuzumab, pertuzumab, and optionally paclitaxel. In some embodiments, said combination is administered according to any uses as detailed herein.

Also provided is a use of a combination in the manufacture of a medicament for preventing or delaying development of resistance of a tumor (e.g., breast cancer) to a therapy containing a HER2-targeted therapy, wherein said combination comprises inavolisib, trastuzumab, pertuzumab, and optionally paclitaxel. In some embodiments, said combination is administered according to any uses as detailed herein.

### BRIEF DESCRIPTION OF THE DRAWINGS

FIG. 1 shows the viability IC₅₀ values determined by quantifying ATP from breast tumor lines: HER2-positive PIK3CA-mutant (n=6), HER2-positive PIK3CA-wild-type (n=4), HER2-negative PIK3CA-mutant (n=10), and HER2-negative PIK3CA-wild-type (n=20) for 5 days with increasing concentration of inavolisib or alpelisib.

FIG. 2 shows the tumor growth curve from KPL-4 (HER2+, PIK3CA H1047R) xenograft treated with vehicle, inavolisib, trastuzumab+pertuzumab, or a combination of inavolisib, trastuzumab and pertuzumab.

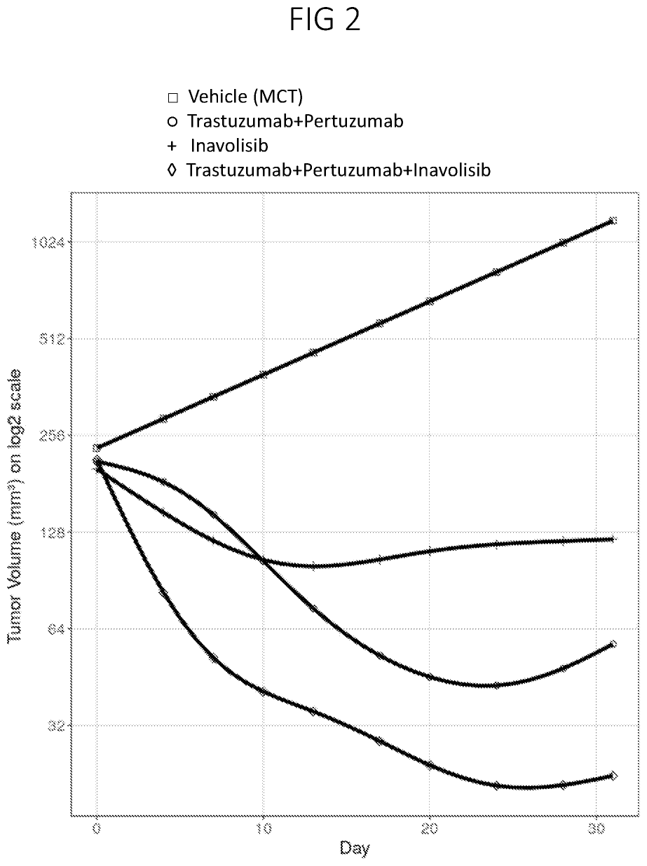

### DETAILED DESCRIPTION OF THE INVENTION

### Definitions

The words “comprise,” “comprising,” “include,” “including,” and “includes” when used in this specification and claims are intended to specify the presence of stated features, integers, components, or steps, but they do not preclude the presence or addition of one or more other features, integers, components, steps, or groups thereof.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the drug may prevent growth and/or kill existing cancer cells, it may be cytostatic and/or cytotoxic. For cancer therapy, efficacy can be measured, for example, by assessing the time to disease progression (TTP) and/or determining the response rate (RR).

“Time to progression” or “TTP” refers to the time from randomization until objective tumor progression.

“Objective response rate” or “ORR” refers to the proportion of patients with a confirmed complete response or partial response on two consecutive occasions≥4 weeks apart, as determined by the investigator according to RECIST v1.1.

“Best overall response rate” or “BOR” refers to the proportion of patients with a CR or PR, as determined by the investigator according to RECIST v1.1.

“Duration of response” or “DOR” refers to the time from the first occurrence of a documented objective response to disease progression, as determined by the investigator according to RECIST v1.1, or death from any cause, whichever occurs first.

“Clinical benefit rate” or “CBR” refers to the proportion of patients with complete response, partial response, and/or stable disease for at least ≥24 weeks, as determined according to RECIST v1.1.

“Overall survival” or “OS” refers to the time from enrollment to death from any cause.

“Time to deterioration (TTD) in pain” refers to the time from randomization to the first documentation of a ≥2-point increase from baseline on the “worst pain” item from the Brief Pain Inventory—Short Form (BPI-SF).

“Time to deterioration (TTD) in Physical Function” refers to the time from randomization to the first documentation of a ≥10-point decrease from baseline in the European Organisation for Research and Treatment of Cancer Quality of Life—Core 30 Questionnaire (EORTC QLQ-C30) Physical Function scale (items 1-5).

“Time to deterioration (TTD) in Role Function” refers to the time from randomization to the first documentation of a ≥10-point decrease from baseline in the EORTC QLQ-C30 Role Function scale (items 6 and 7).

“Time to deterioration (TTD) in global health status (GHS)/health-related quality of life (HRQoL)” refers to the time from randomization to the first documentation of a ≥10-point decrease from baseline in the EORTC QLQ-30 GHS/HRQoL scale (items 29 and 30).

“Progression free survival” or “PFS” refers to the time from enrollment to the date of the first recorded occurrence of disease progression, as determined by the investigator using RECIST v1.1 or death from any cause, whichever occurs first.

“Complete response” or “CR” refers to the disappearance of all target lesions and non-target lesions and (if applicable) normalization of tumor marker level.

“Partial response”, “PR” or “Non-CR/Non-PrD” refers to persistence of one or more non-target lesions and/or (if applicable) maintenance of tumor marker level above the normal limits. A PR can also refer to ≥30% decrease in sum of diameters of target lesions, in the absence of CR, new lesions, and unequivocal progression in non-target lesions.

“Progressive disease” or “PrD” refers to ≥20% increase in sum of diameters of target lesions, unequivocal progression in non-target lesions, and/or appearance of new lesions.

“Stable disease” or “SD” refers to neither sufficient shrinkage to qualify for CR or PR nor sufficient increase growth of tumor to qualify for PrD.

An “administration period” or “cycle” refers to a period of time comprising administration of one or more agents described herein and an optional period of time comprising no administration of one or more of the agents described herein. For example, a cycle can be 28 days in total length and include administration of one or more agents for 21 days and a rest period of 7 days. A “rest period” refers to a period of time where at least one of the agents described herein are not administered. In one embodiment, a rest period refers to a period of time where none of the agents described herein are administered. In one embodiment, a cycle does not include any rest period.

A “dosing regimen” refers to a period of administration of the agents described herein comprising one or more cycles, where each cycle can include administration of the agents described herein at different times or in different amounts.

“QD” refers to administration of a compound once daily.

A graded adverse event refers to the severity grading scale as established for by NCI CTCAE. In one embodiment, the adverse event is graded in accordance with the table below.

| Grade | Severity |
|---|---|
| 1 | Mild; asymptomatic or mild symptoms; clinical or diagnostic |
| | observations only; or intervention not indicated |
| 2 | Moderate; minimal, local, or non-invasive intervention indicated; |
| | or limiting age-appropriate instrumental activities of daily |
| | living |
| 3 | Severe or medically significant, but not immediately life- |
| | threatening; hospitalization or prolongation of hospitalization |
| | indicated; disabling; or limiting self-care activities of daily |
| | living |
| 4 | Life-threatening consequences or urgent intervention indicated |
| 5 | Death related to adverse event |

The term “detection” includes any means of detecting, including direct and indirect detection.

The term “prognosis” is used herein to refer to the prediction of the likelihood of cancer-attributable death or progression, including, for example, recurrence, metastatic spread, and drug resistance, of a neoplastic disease, such as cancer.

The term “prediction” (and variations such as predicting) is used herein to refer to the likelihood that a patient will respond either favorably or unfavorably to a drug or set of drugs. In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The term “increased resistance” to a particular therapeutic agent or treatment option, when used in accordance with the invention, means decreased response to a standard dose of the drug or to a standard treatment protocol.

“Response” can be assessed using any endpoint indicating a benefit to the patient, including, without limitation, (1) inhibition, to some extent, of tumor growth, including slowing down or complete growth arrest; (2) reduction in the number of tumor cells; (3) reduction in tumor size; (4) inhibition (e.g., reduction, slowing down or complete stopping) of tumor cell infiltration into adjacent peripheral organs and/or tissues; (5) inhibition (e.g., reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

A “biomarker” is a characteristic that is objectively measured and evaluated as an indicator of normal biological processes, pathogenic processes, or pharmacological responses to a therapeutic intervention. Biomarkers may be of several types: predictive, prognostic, or pharmacodynamics (PD). Predictive biomarkers predict which patients are likely to respond or benefit from a particular therapy. Prognostic biomarkers predict the likely course of the patient's disease and may guide treatment. Pharmacodynamic biomarkers confirm drug activity, and enables optimization of dose and administration schedule.

“Change” or “modulation” of the status of a biomarker, including a PIK3CA mutation or set of PIK3CA mutations, as it occurs in vitro or in vivo is detected by analysis of a biological sample using one or more methods commonly employed in establishing pharmacodynamics (PD), including: (1) sequencing the genomic DNA or reverse-transcribed PCR products of the biological sample, whereby one or more mutations are detected; (2) evaluating gene expression levels by quantitation of message level or assessment of copy number; and (3) analysis of proteins by immunohistochemistry (IHC), immunocytochemistry, ELISA, or mass spectrometry whereby degradation, stabilization, or post-translational modifications of the proteins such as phosphorylation or ubiquitination is detected.

A “chemotherapeutic agent” is a biological (large molecule) or chemical (small molecule) compound useful in the treatment of cancer, regardless of mechanism of action.

The term “package insert” is used to refer to instructions customarily included in commercial packages of therapeutic products, that contain information about the indications, usage, dosage, administration, contraindications and/or warnings concerning the use of such therapeutic products.

The phrase “pharmaceutically acceptable salt” as used herein, refers to pharmaceutically acceptable organic or inorganic salts of a compound of the invention. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate”, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion.

The desired pharmaceutically acceptable salt may be prepared by any suitable method available in the art. For example, treatment of the free base with an inorganic acid, such as hydrochloric acid, hydrobromic acid, sulfuric acid, nitric acid, methanesulfonic acid, phosphoric acid and the like, or with an organic acid, such as acetic acid, maleic acid, succinic acid, mandelic acid, fumaric acid, malonic acid, pyruvic acid, oxalic acid, glycolic acid, salicylic acid, a pyranosidyl acid, such as glucuronic acid or galacturonic acid, an alpha hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like. Acids which are generally considered suitable for the formation of pharmaceutically useful or acceptable salts from basic pharmaceutical compounds are discussed, for example, by P. Stahl et al, Camille G. (eds.) Handbook of Pharmaceutical Salts. Properties, Selection and Use. (2002) Zurich: Wiley-VCH; S. Berge et al, Journal of Pharmaceutical Sciences (1977) 66(1) 1 19; P. Gould, International J. of Pharmaceutics (1986) 33 201 217; Anderson et al, The Practice of Medicinal Chemistry (1996), Academic Press, New York; Remington's Pharmaceutical Sciences, 18^{th} ed., (1995) Mack Publishing Co., Easton PA; and in The Orange Book (Food & Drug Administration, Washington, D.C. on their website). These disclosures are incorporated herein by reference thereto.

The phrase “pharmaceutically acceptable” indicates that the substance or composition must be compatible chemically and/or toxicologically, with the other ingredients comprising a formulation, and/or the patient being treated therewith.

The term “synergistic” as used herein refers to a therapeutic combination which is more effective than the additive effects of the two or more single agents. A determination of a synergistic interaction between a compound of inavolisib or a pharmaceutically acceptable salt thereof, and one or more chemotherapeutic agent may be based on the results obtained from the assays described herein. The results of these assays can be analyzed using the Chou and Talalay combination method and Dose-Effect Analysis with CalcuSyn® software in order to obtain a Combination Index (Chou and Talalay, 1984, Adv. Enzyme Regul. 22:27-55). The combinations provided by this invention have been evaluated in several assay systems, and the data can be analyzed utilizing a standard program for quantifying synergism, additivism, and antagonism among anticancer agents described by Chou and Talalay, in “New Avenues in Developmental Cancer Chemotherapy,” Academic Press, 1987, Chapter 2. Combination Index values less than 0.8 indicates synergy, values greater than 1.2 indicate antagonism and values between 0.8 and 1.2 indicate additive effects. The combination therapy may provide “synergy” and prove “synergistic”, i.e., the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation therapy, a synergistic effect may be attained when the compounds are administered or delivered sequentially, e.g., by different injections in separate syringes or in separate pills or tablets. In general, during alternation therapy, an effective dosage of each active ingredient is administered sequentially, i.e., serially, whereas in combination therapy, effective dosages of two or more active ingredients are administered together. Combination effects were evaluated using both the BLISS independence model and the highest single agent (HSA) model (Lehár et al. 2007, Molecular Systems Biology 3:80). BLISS scores quantify degree of potentiation from single agents and a BLISS score>0 suggests greater than simple additivity. An HSA score>0 suggests a combination effect greater than the maximum of the single agent responses at corresponding concentrations.

### Clinical Compounds

Inavolisib (a.k.a. GDC-0077):

Inavolisib is a potent, orally bioavailable, clinical-stage, selective inhibitor of the Class I PI3K alpha isoform (PI3Kα), with >300-fold less potent biochemical inhibition for other Class I PI3K beta, delta, and gamma isoforms and increased potency in tumor cells bearing mutant PI3K over wild type (WT) PI3K cells (Braun, M. et al “Discovery of GDC-0077: A highly selective inhibitor of PI3K-alpha that induces degradation of mutant-p110 alpha protein” Abstracts of Papers, 254th ACS National Meeting & Exposition, Washington, DC, USA, Aug. 20-24, 2017, MEDI-22; Garland, K. et al “Discovery of novel class of alpha selective PI3K inhibitors” Abstracts of Papers, 254th ACS National Meeting & Exposition, Washington, DC, USA, Aug. 20-24, 2017, MEDI-103; Hong, R. et al “GDC-0077 is a selective PI3K alpha inhibitor that demonstrates robust efficacy in PIK3CA mutant breast cancer models as a single agent and in combination with standard of care therapies” 2017 San Antonio Breast Cancer Symposium, Dec. 5-9, 2017, San Antonio, TX, Abstract Publication Number: PD4-14; Edgar, K. et al “Preclinical characterization of GDC-0077, a specific PI3K alpha inhibitor in early clinical development” Cancer Research 77(13 Supplement): Abstract 156 July 2017).

Inavolisib, CAS Registry Number 2060571-02-8, Genentech, Inc., U.S. Pat. No. 9,650,393; named as (S)-2-((2-((S)-4-(difluoromethyl)-2-oxooxazolidin-3-yl)-5,6-dihydrobenzo[f]imidazo[1,2-d][1,4]oxazepin-9- yl)amino)propanamide, has the structure:

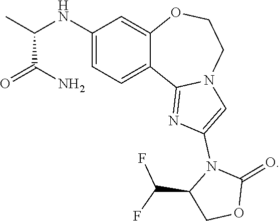 [SMILES: C[C@H](Nc1ccc2c(c1)OCCn1cc(N3C(=O)OC[C@H]3C(F)F)nc1-2)C(N)=O]

Inavolisib is also known as GDC-0077, RG6114, RO7113755, or chemical name (2S)-2-[[2-[(4S)-4-(Difluoromethyl)-2-oxo-3-oxazolidinyl]-5,6-dihydroimidazo[1,2-d][1,4]benzoxazepin-9-yl]amino]propanamide.

GDC-0077 exerts its activity by binding to the ATP binding site of PI3K, thereby inhibiting the phosphorylation of membrane-bound 4,5-phosphatidylinositol bisphosphate (PIP₂) to 3,4,5-phosphatidylinositol triphosphate (PIP₃). Inhibiting the phosphorylation of PIP₂ to PIP₃ decreases downstream activation of AKT and pS6, resulting in decreased cellular proliferation, metabolism, and angiogenesis. Nonclinical studies demonstrate that GDC-0077 specifically degrades mutant p110 alpha, inhibits proliferation and induces apoptosis of PIK3CA-mutant breast cancer cell lines, inhibits tumor growth in human breast xenograft models harboring PIK3CA mutations, and reduces downstream PI3K-pathway markers, including phosphorylated form of protein kinase B (pAKT), PRAS40 phosphorylated at Threonine 246 (pPRAS40), and S6RP phosphorylated at Serine 235/236 (pS6RP).

### Trastuzumab:

Trastuzumab (CAS 180288-69-1, HERCEPTIN®, huMAb4D5-8, rhuMAb HER2, Genentech) is a recombinant DNA-derived, IgG1 kappa, monoclonal antibody that is a humanized version of a murine anti-HER2 antibody (4D5) that selectively binds with high affinity in a cell-based assay (Kd=5 nM) to the extracellular domain of HER2 (U.S. Pat. Nos. 5,677,171; 5,821,337; 6,054,297; 6,165,464; 6,339,142; 6,407,213; 6,639,055; 6,719,971; 6,800,738; 7,074,404; Coussens et al (1985) Science 230:1132-9; Slamon et al (1989) Science 244:707-12; Slamon et al (2001) New Engl. J. Med. 344:783-792). Trastuzumab has been shown, in both in vitro assays and in animals, to inhibit the proliferation of human tumor cells that overexpress HER2 (Hudziak et al (1989) Mol Cell Biol 9:1165-72; Lewis et al (1993) Cancer Immunol Immunother; 37:255-63; Baselga et al (1998) Cancer Res. 58:2825-2831). Trastuzumab is a mediator of antibody-dependent cellular cytotoxicity, ADCC (Lewis et al (1993) Cancer Immunol Immunother 37(4):255-263; Hotaling et al (1996) [abstract]. Proc. Annual Meeting Am Assoc Cancer Res; 37:471; Pegram M D, et al (1997) [abstract]. Proc Am Assoc Cancer Res; 38:602; Sliwkowski et al (1999) Seminars in Oncology 26(4), Suppl 12:60-70; Yarden Y. and Sliwkowski, M. (2001) Nature Reviews: Molecular Cell Biology, Macmillan Magazines, Ltd., Vol. 2:127-137).

HERCEPTIN® (trastuzumab) was approved in 1998 for the treatment of patients with HER2-overexpressing metastatic breast cancers (Baselga et al, (1996) J. Clin. Oncol. 14:737-744) that have received extensive prior anti-cancer therapy, and has since been used in over 300,000 patients (Slamon D J, et al. N Engl J Med 2001; 344:783-92; Vogel C L, et al. J Clin Oncol 2002;20:719-26; Marty M, et al. J Clin Oncol 2005; 23:4265-74; Romond E H, et al. T N Engl J Med 2005; 353:1673-84; Piccart-Gebhart M J, et al. N Engl J Med 2005; 353:1659-72; Slamon D, et al. [abstract]. Breast Cancer Res Treat 2006, 100 (Suppl 1): 52). In 2006, the FDA approved HERCEPTIN® (trastuzumab, Genentech Inc.) as part of a treatment regimen containing doxorubicin, cyclophosphamide and paclitaxel for the adjuvant treatment of patients with HER2-positive, node-positive breast cancer.

HERCEPTIN HYLECTA™ (trastuzumab and hyaluronidase-oysk) is a combination of trastuzumab and recombinant human hyaluronidase (an endoglycosidase, tissue permeability modifier administered by subcutaneous fluid administration), and has been approved by the U.S. FDA for treatment of HER2-overexpressing breast cancer.

### Pertuzumab:

Pertuzumab (also known as recombinant humanized monoclonal antibody 2C4, rhuMAb 2C4, PERJETA®, Genentech, Inc, South San Francisco) represents the first in a new class of agents known as HER dimerization inhibitors (HDI) and functions to inhibit the ability of HER2 to form active heterodimers or homodimers with other HER receptors (such as EGFR/HER1, HER2, HER3 and HER4). See, for example, Harari and Yarden Oncogene 19:6102-14 (2000); Yarden and Sliwkowski. Nat Rev Mol Cell Biol 2:127-37 (2001); Sliwkowski Nat Struct Biol 10:158-9 (2003); Cho et al. Nature 421:756-60 (2003); and Malik et al. Pro Am Soc Cancer Res 44:176-7 (2003).

PERJETA® (pertuzumab) was approved in 2012 for the treatment of patients with advanced or late-stage (metastatic) HER2-positive breast cancer. On Sep. 30, 2013, the U.S. Food and Drug Administration granted accelerated approval to PERJETA® (pertuzumab) as part of a complete treatment regimen for patients with early stage breast cancer (EBC) before surgery (neoadjuvant setting). PERJETA® is the first FDA-approved drug for the neoadjuvant treatment of breast cancer.

PHESGO® (pertuzumab, trastuzumab, and hyaluronidase-zzxf) contains a fixed-dose combination of pertuzumab and trastuzumab with hyaluronidase for injection under the skin, and has been approved by U.S. FDA in 2020 for the treatment of early and metastatic HER2-positive breast cancer. PHESGO can be administered by subcutaneous (SC; under the skin) injection in combination with intravenous (IV) chemotherapy.

Pertuzumab inhibits ligand-initiated intracellular signaling through major signal pathways, including PI3K, which can result in cell growth arrest and apoptosis. In addition, Both trastuzumab and pertuzumab mediate antibody-dependent cellular cytotoxicity (ADCC). Overall, the combination of pertuzumab and trastuzumab is well tolerated, without significant increase in left ventricular systolic dysfunction.

### Fulvestrant:

Fulvestrant is an ER antagonist and an effective treatment for postmenopausal patients with HR+breast cancer that is relatively well tolerated. The expected toxicities for GDC-0077 and fulvestrant are not overlapping. It is important to test GDC-0077 in combination with both letrozole and fulvestrant, as these endocrine therapies have different mechanisms of action, different PK properties, and different potential for drug-drug interactions (DDIs) with inavolisib.

Fulvestrant (FASLODEX®, AstraZeneca, CAS Reg. No. 129453-61-8) is approved by the FDA for treatment of hormone receptor-positive (HR+) metastatic breast cancer in postmenopausal women with disease progression following anti-estrogen therapy (Kansra (2005) Mol Cell Endocrinol 239(1-2):27-36; Flemming et al (2009) Breast Cancer Res Treat. May; 115(2):255-68; Valachis et al (2010) Crit Rev Oncol Hematol. March;73(3):220-7). Fulvestrant is an estrogen receptor (ER) antagonist with no agonist effects, which works both by down-regulating and by degrading the estrogen receptor (Croxtall (2011) Drugs 71(3):363-380). Fulvestrant is also a selective estrogen receptor down-regulator (SERD).

Fulvestrant is named as (7α,17β)-7-{9-[(4,4,5,5,5-pentafluoropentyl)sulfinyl]nonyl}estra-1,3,5(10)-triene-3,17-diol and has the structure:

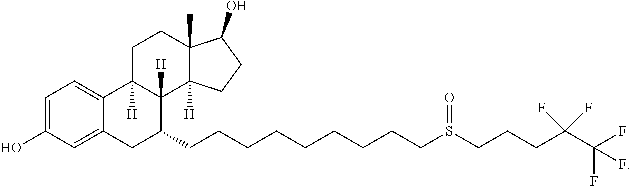

Fulvestrant belongs to a class of reversible steroidal ER antagonists that directly competes with estrogen for ER binding and is devoid of the partial agonist properties of tamoxifen. Upon binding to ER, it blocks estrogen signaling and increases the degradation of ER protein. The affinity of fulvestrant for the ER is approximately 100-fold greater than that of tamoxifen (Howell et al. (2000) Cancer 89:817-25). Fulvestrant (250 mg once monthly) was approved by the FDA in 2002 and by the EMA in 2004 for the treatment of HR-positive MBC in postmenopausal women with disease progression following anti-estrogen therapy. In multicenter Phase III studies, fulvestrant was found to be at least equivalent to anastrozole (a non-steroidal AI) in the second-line setting (Howell et al. (2002) J Clin Oncol 20:3396-3403; Osborne C K, et al (2002) J Clin Oncol 20:3386-95). Fulvestrant is also as active as tamoxifen for the first-line treatment of advanced breast cancer (Howell et al. (2004) J Clin Oncol 22:1605-1613) and displays a level of activity in patients in the post-AI metastatic disease setting similar to that of the non-steroidal AI exemestane (Chia et al. (2008) J Clin Oncol 26:1664-1670). High-dose fulvestrant (500 mg once monthly) has been demonstrated to be at least as effective as anastrozole in terms of clinical benefit rate (CBR) and overall response rate and to be associated with significantly longer time to progression for the first-line treatment of women with advanced HR-positive breast cancer (Robertson et al. (2009) J Clin Oncol 27:4530-4535). High-dose fulvestrant recently demonstrated superior progression-free survival (PFS) in women with ER-positive advanced breast cancer treated with 500 mg versus patients treated with 250 mg (Di Leo et al. (2010) J Clin Oncol 28:4594-4600). Fulvestrant (250 mg and 500 mg) was well tolerated in these studies and produced fewer estrogenic effects than did tamoxifen and resulted in less arthralgia than did the AI anastrozole (Osborne et al. (2002) J Clin Oncol 20:3386-3395). These results led to the approval of 500 mg fulvestrant given once a month as the currently approved recommended dose in the United States and the European Union (in 2010) for postmenopausal women whose disease has spread after treatment with an AI. These studies demonstrate that fulvestrant is an important treatment option for patients with advanced breast cancer and, as such, is considered appropriate control therapy for the present study.

### Letrozole:

Letrozole is an effective treatment for postmenopausal patients with HR+breast cancer that is relatively well tolerated. The expected toxicities for inavolisib and letrozole are not overlapping. Letrozole (FEMARA®, Novartis Pharm.) is an oral non-steroidal aromatase inhibitor for the treatment of hormonally-responsive breast cancer after surgery (Bhatnagar et al (1990) J Steroid Biochem. and Mol. Biol. 37:1021; Lipton et al (1995) Cancer 75:2132; Goss, P. E. and Smith, R. E. (2002) Expert Rev. Anticancer Ther. 2:249-260; Lang et al (1993) The Journal of Steroid Biochem. and Mol. Biol. 44 (4-6):421-8; EP 236940; U.S. Pat. No. 4,978,672). FEMARA® is approved by the FDA for the treatment of local or metastatic breast cancer that is hormone receptor positive (HR+) or has an unknown receptor status in postmenopausal women.

Letrozole is named as 4,4′-((1H-1,2,4-triazol-1-yl)methylene)dibenzonitrile (CAS Reg. No. 112809-51-5), and has the structure:

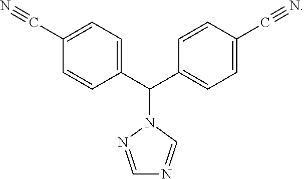 [SMILES: N#Cc1ccc(C(c2ccc(C#N)cc2)n2cncn2)cc1]

### Paclitaxel:

Paclitaxel is a chemotherapy medication approved for the treatment of a number of types of cancers (for example, ovarian cancer, breast cancer, lung cancer, etc.), as a monotherapy or in combination with other anti-cancer agents. Paclitaxel is named as 5β,20-Epoxy-1,2α,4,7β,10β,13α-hexahydroxytax-11-en-9-one 4,10- diacetate 2-benzoate 13-ester with (2R,3S)-N-benzoyl-3-phenylisoserine, and has the structure:

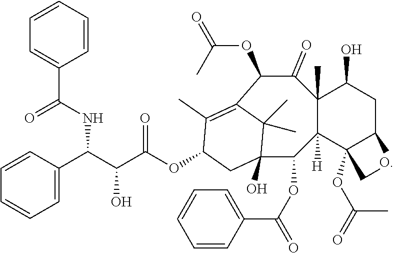

### Combination Therapy

In human epidermal growth factor receptor 2-positive (HER2+) breast cancer, dysregulation of the PI3K/AKT/mTOR pathway, in the form of activating mutations and other aberrations, has been identified as a possible mechanism of resistance to HER2-targeted therapies. As such, continuous inhibition of the HER2 pathway, along with co-targeting of the PI3K/AKT/mTOR pathway, may restore sensitivity to HER2-targeted therapies. Addition of a PI3K inhibitor to trastuzumab and pertuzumab may improve outcomes for patients with PIK3CA-mutant HER2+ breast cancer.

Provided herein are combinations or combination therapies comprising inavolisib (GDC-0077) and a HER2-targeted therapy. In some embodiments, the HER2-targeted therapy is trastuzumab, pertuzumab, or a combination of trastuzumab and pertuzumab. In one embodiment, the combination or combination therapy comprises inavolisib, trastuzumab and pertuzumab. In one embodiment, the combination or combination therapy further comprises hyaluronidase. In one embodiment, the combination or combination therapy comprises inavolisib, trastuzumab and hyaluronidase-oysk, and pertuzumab. In one embodiment, the combination or combination therapy comprises inavolisib, pertuzumab, trastuzumab and hyaluronidase-zzxf.

The combinations or combination therapies described herein can be provided as a kit comprising one or more of the agents for administration. In one embodiment, the kit includes inavolisib and trastuzumab. In one embodiment, the kit includes inavolisib, trastuzumab and hyaluronidase-oysk. In one embodiment, the kit includes inavolisib and pertuzumab. In another embodiment, the kit includes inavolisib, trastuzumab and pertuzumab. In another embodiment, the kit includes inavolisib and a fixed dose combination of trastuzumab and pertuzumab. In another embodiment, the kit includes inavolisib, pertuzumab, trastuzumab, and hyaluronidase-zzxf. In one embodiment, the agents of the combination or combination therapy described herein are supplied in a kit in a form ready for administration. Kits described herein can include instructions such as package inserts. In one embodiment, the instructions are package inserts—one for each agent in the kit.

Further provided are kits for carrying out the methods detailed herein, which comprises a phamaceutical composition or a combination therapy described herein and instructions for use in the treatment of breast cancer.

Kits generally comprise suitable packaging. The kits may comprise one or more containers comprising any phamaceutical composition described herein. Each component (if there is more than one component) can be packaged in separate containers or some components can be combined in one container where cross-reactivity and shelf life permit. One or more components of a kit may be sterile and/or may be contained within sterile packaging.

### Methods

HER2 overexpression is an important prognostic and predictive biomarker in metastatic breast cancer (Pauletti et al. 2000). In the phase III study of trastuzumab plus docetaxel with or without pertuzumab in patients with 1L HER2+ mBC, above-median (high) HER2 protein expression by immunohistochemistry and above-median (high) HER2 mRNA expression by qRT-PCR (quantitative reverse transcription polymerase chain reaction) were significantly associated with better prognosis in this patient population (HR 0.83 [p =0.05] and HR 0.77 [p=0.008], respectively) (Baselga, et al. 2014). Frequent PIK3CA mutations are also observed in patients with HER2 expression, and there are differences in prevalence of PIK3CA mutations. PIK3CA mutations are observed in nearly twice the number of patients whose tumors had focal, heterogeneous HER2 expression (42%) relative to the number of patients whose tumors had robust homogenous HER2 expression (24%) by IHC (Perez et al. 2019). Thus it is beneficial to treat HER2+breast cancer with a combination of a mutant PI3Kα inhibitor and a HER2-targeted therapy.

All PI3Kα inhibitors are not equal. Inavolisib (GDC-0077) is especially advantageous in inhibiting growth of HER2+ PIK3CA-mutant cancer cells over another clinically relevant PI3Kα inhibitor alpelisib (a.k.a. BYL719). In a study comparing efficacy of inavolisib with alpelisib, the results showed a significant difference between the sensitivity of inavolisib and alpelisib in HER2-amplified (˜20-fold difference between the mean IC50 values) versus HER2-negative cell lines (6-fold difference between two inhibitors). Both inhibitors were not differentiated in PIK3CA-WT cell lines regardless of HER2 status.

Provided herein are methods of treating HER2 positive cancer. In one embodiment, the method comprises treating HER2-positive breast cancer in a patient having locally advanced or metastatic PIK3CA-mutated HER2-positive breast cancer, by administering to the patient a combination therapy that includes inavolisib and a HER2-targeted therapy (e.g., trastuzumab, pertuzumab, or a combination of trastuzumab and pertuzumab).

Also provided are methods of treating locally advanced or metastatic PIK3CA-mutated (or PIK3CA mutant) HER2-positive breast cancer in a patient comprising administering a therapeutically effective amount of inavolisib (GDC-0077), or a pharmaceutically acceptable salt thereof, and a HER2-targeted therapy (e.g., trastuzumab, pertuzumab, or a combination of trastuzumab and pertuzumab). In some embodiments, the HER2-targeted therapy comprises trastuzumab. In some embodiments, the HER2-targeted therapy comprises trastuzumab and hyaluronidase-oysk. In some embodiments, the HER2-targeted therapy comprises pertuzumab. In some embodiments, the HER2-targeted therapy comprises trastuzumab and pertuzumab. In some embodiments, the HER2-targeted therapy comprises a fixed dose combination of trastuzumab and pertuzumab. In some embodiments, the HER2-targeted therapy comprises pertuzumab, trastuzumab and hyaluronidase-zzxf. In some embodiments, the method further comprises administering paclitaxel.

In some embodiments, provided is a method of treating HER2-positive breast cancer in a patient having locally advanced or metastatic PIK3CA-mutated HER2-positive breast cancer, the method comprising administering to the patient a combination therapy comprising: inavolisib, trastuzumab and pertuzumab; wherein said combination therapy is administered over a 21-day cycle. In some embodiments, the combination therapy further comprises paclitaxel.

In some embodiments, provided is a method of treating HER2-positive breast cancer in a patient having locally advanced or metastatic PIK3CA-mutated HER2-positive breast cancer, the method comprising administering to the patient a combination therapy comprising a dosing regimen comprising:
a. administering inavolisib QD on days 1-21 of a first 21-day cycle;
b. administering trastuzumab on day 1 of a first 21-day cycle; and
c. administering pertuzumab on day 1 of a first 21-day cycle.

In some of these embodiments, the method further comprises one or more additional 21-day cycles comprising:
a. administering inavolisib on days 1-21 of each additional 21-day cycle;
b. administering trastuzumab on day 1 of each additional 21-day cycle; and
c. administering pertuzumab on day 1 of each additional 21-day cycle.

In some embodiments, provided is a method of treating HER2-positive breast cancer in a patient having locally advanced or metastatic PIK3CA-mutated HER2-positive breast cancer, the method comprising administering to the patient a combination therapy comprising a dosing regimen comprising:
a. administering paclitaxel on days 1, 8 and 15 of a first 21-day cycle;
b. administering inavolisib QD on days 2-21 of a first 21-day cycle;
c. administering trastuzumab on day 2 of a first 21-day cycle; and
d. administering pertuzumab on day 2 of a first 21-day cycle.

In some of these embodiments, the method further comprises one or more additional 21-day cycles (e.g., one, two, three, four or five additional 21-day cycles) comprising:
a. administering paclitaxel on days 1, 8 and 15 of each additional 21-day cycle;
b. administering inavolisib on days 1-21 of each additional 21-day cycle;
c. administering trastuzumab on day 1 of each additional 21-day cycle; and
d. administering pertuzumab on day 1 of each additional 21-day cycle.

Depending on dose-limiting toxicities (DLTs) observed in the patient, inavolisib may be administered on a 6/1 or 5/2 dosing regimen. Inavolisib is given for 6 days in a week with 1 day off, or 5 days in a week with 2 days off.

In some embodiments, provided is a method of treating HER2-positive breast cancer in a patient having locally advanced or metastatic PIK3CA-mutated HER2-positive breast cancer, the method comprising administering to the patient a combination therapy comprising a dosing regimen comprising:
a. administering paclitaxel on days 1, 8 and 15 of a first 21-day cycle;
b. administering inavolisib QD on days 2-7, 9-14 and 16-21 of a first 21-day cycle;
c. administering trastuzumab on day 2 of a first 21-day cycle; and
d. administering pertuzumab on day 2 of a first 21-day cycle.

In some of these embodiments, the method further comprises one or more additional 21-day cycles (e.g., one, two, three, four or five additional 21-day cycles) comprising:
a. administering paclitaxel on days 1, 8 and 15 of each additional 21-day cycle;
b. administering inavolisib on days 2-7, 9-14 and 16-21 of each additional 21-day cycle;
c. administering trastuzumab on day 1 of each additional 21-day cycle; and
d. administering pertuzumab on day 1 of each additional 21-day cycle.

In some embodiments, provided is a method of treating HER2-positive breast cancer in a patient having locally advanced or metastatic PIK3CA-mutated HER2-positive breast cancer, the method comprising administering to the patient a combination therapy comprising a dosing regimen comprising:
a. administering paclitaxel on days 1, 8 and 15 of a first 21-day cycle;
b. administering inavolisib QD on days 3-7, 10-14 and 17-21 of a first 21-day cycle;
c. administering trastuzumab on day 2 of a first 21-day cycle; and
d. administering pertuzumab on day 2 of a first 21-day cycle.

In some of these embodiments, the method further comprises one or more additional 21-day cycles (e.g., one, two, three, four or five additional 21-day cycles) comprising:
a. administering paclitaxel on days 1, 8 and 15 of each additional 21-day cycle;
b. administering inavolisib on days 3-7, 10-14 and 17-21 of each additional 21-day cycle;
c. administering trastuzumab on day 1 of each additional 21-day cycle; and
d. administering pertuzumab on day 1 of each additional 21-day cycle.

After receiving up to six cycles of combination therapy comprising paclitaxel, inavolisib, pertuzumab and trastuzumab (induction therapy), the patient may continue receiving additional cycles of a combination therapy comprising inavolisib, pertuzumab and trastuzumab (without paclitaxel) (maintenance therapy) until disease progression or unacceptable related toxicity. During maintenance therapy, patients with HR-positive/HER2-positive metastatic breast cancer may be treated with an endocrine therapy (e.g., letrozole or fulvestrant).

In some of these embodiments, the method comprising administering to the patient an induction therapy comprising paclitaxel, inavolisib, pertuzumab and trastuzumab in a regimen comprising a first 21-day cycle as detailed herein and up to five additional 21-day cycles as detailed herein, further comprises a maintenance therapy comprising inavolisib, pertuzumab and trastuzumab (without paclitexel) in a regimen comprising:
a. administering inavolisib on days 1-21 of each additional 21-day cycle;
b. administering trastuzumab on day 1 of each additional 21-day cycle; and
c. administering pertuzumab on day 1 of each additional 21-day cycle.

In one embodiment, the method includes a combination therapy comprising inavolisib and a HER2-targeted therapy (e.g., trastuzumab, pertuzumab, or a combination of trastuzumab and pertuzumab). In one embodiment, the method includes a combination therapy comprising inavolisib, trastuzumab and pertuzumab administered in accordance with a dosing regimen described herein. In one embodiment, the method includes a combination therapy comprising paclitaxel, inavolisib, trastuzumab and pertuzumab administered in accordance with a dosing regimen described herein.

In a further aspect, provided is a method of inhibiting tumor growth or producing/increasing tumor regression in a patient having locally advanced or metastatic PIK3CA-mutant HER2-positive breast cancer, the method comprising administering to the patient a combination therapy according to the methods detailed herein.

Agents described herein can be administered in accordance with a package insert. In one embodiment of the methods described herein, agents can be administered in an effective amount as described herein. In some embodiments, trastuzumab and/or pertuzumab, where applicable, is administered at its approved dosage by an approved route of administration.

Agents in a combination therapy detailed herein may be administered simultaneously or sequencially. Two of the triplets of a combination therapy may be administer simultaneously while the third agent may be adminstered before or after. For example, in one embodiment of the methods described herein, administration of inavolisib occurs before administration of another agent (e.g. pertuzumab or trastuzumab). In one embodiment of the methods described herein, inavolisib is administered first followed by pertuzumab and then trastuzumab. In another embodiment, inavolisib is administered before or after a fixed dose combination of pertuzumab and trastuzumab, for example, a fixed dose combination of pertuzumab, trastuzumab and hyaluronidase-zzxf. In some embodiments, paclitaxel is administered before the triplet combination therapy is administered in any orders as detailed herein.

In some embodiments, inavolisib is administered at an amount of 3, 6 or 9 mg, e.g., in one or more oral tablets. In some embodiments, inavolisib is adminstered orally at a 9 mg daily dose. In some of these embodiments, inavolisib is administered at an amount of 9 mg, e.g., in an oral tablet. In some embodiments, inavolisib is adminstered orally at a 6 mg daily dose, e.g., in one or more oral tablets.

In some embodiments, trastuzumab is administered by intravenous (IV) infusion. In some embodiments, trastuzumab is administered at a loading dose of 8 mg/kg for the first 21-day cycle. In some embodiments, trastuzumab is administered at a dose of 6 mg/kg for each additional cycle. In some embodiments, trastuzumab is administered by subcutaneous (SC) injection. In some embodiments, trastuzumab is administered by subcutaneous (SC) injection with hyaluronidase-oysk.

In some embodiments, pertuzumab is administered by intravenous (IV) infusion. In some embodiments, pertuzumab is administered at a loading dose of 840 mg for the first 21-day cycle. In some embodiments, pertuzumab is administered at a dose of 840 mg for each additional cycle. In some embodiments, pertuzumab is administered by subcutaneous (SC) injection. In some embodiments, pertuzumab is administered by subcutaneous (SC) injection with trastuzumab and hyaluronidase-zzxf.

In some embodiments, paclitaxel is administered by intravenous (IV) infusion. In some embodiments, paclitaxel is administered at a weekly dose of 80 mg/m². In some embodiments, paclitaxel is administered by intravenous infusion at a dose of 80 mg/m² per week.

In some embodiments, inavolisib is administered at an amount of 9 mg, e.g., in an oral tablet. In some embodiments, trastuzumab is administered at a loading dose of 8 mg/kg for the first 21-day cycle and a dose of 6 mg/kg for each additional 21-day cycle by intravenous (IV) infusion. In some embodiments, pertuzumab is administered at a loading dose of 840 mg for the first 21-day cycle and a dose of 420 mg for each additional 21-day cycle by IV infusion.

In one embodiment, the methods described herein include a combination therapy described herein administered according to a dosing regimen comprising one 21-day cycle. In another embodiment, the methods described herein include a combination therapy described herein administered according to a dosing regimen comprising a first 21-day cycle followed by additional 21-day cycles. In another embodiment, the methods described herein include a combination therapy described herein administered according to a dosing regimen comprising a first 21-day cycle followed by 2 to 30 additional 21-day cycles, or until disease progression or unacceptable toxicity. In yet another embodiment, the methods described herein include an induction therapy described herein administered according to a dosing regimen comprising a first 21-day cycle followed by up to five additional 21-day cycles, or until disease progression or unacceptable toxicity. In another embodiment, the methods described herein include an induction therapy described herein administered according to a dosing regimen comprising a first 21-day cycle followed by up to five additional 21-day cycles, and a maintence therapy described herein administered according to a dosing regimen comprising one or more additional 21-day cycles (e.g., up to 30 additional 21-day cycles), or until disease progression or unacceptable toxicity.

The the efficacy of the combination is measured as a function of progression-free survival (PFS), overall survival (OS), objective response rate (ORR), and other relevant clinical outcome.

In some embodiments, the patient has PIK3CA mutant, HER2 positive, locally advanced or metastatic breast cancer. In some embodiments, the patient is a female patient with histologically documented locally advanced or metastatic PIK3CA-mutant HER2+ breast cancer. The hormone receptor status of the patient may be positive or negative. In some embodiments, the patient has hormone receptor positive (HR+), locally advanced or metastatic PIK3CA-mutated HER2-positive breast cancer. In some embodiments, the patient has hormone receptor nagative (HR−), locally advanced or metastatic PIK3CA-mutated HER2-positive breast cancer.

In some embodiments, the patient has mutant PIK3CA having mutations at one or more of positions 88, 106, 111, 118, 345, 420, 453, 542, 545, 546, 1043, 1047 and 1049. In some embodiments, the patient has mutant PIK3CA having mutations at one or more of H1047, E545, E542, Q546, N345, C420, M1043, G1049, E453, K111, G106, G118, and R88. In some embodiments, the patient has mutant PIK3CA containing one or more mutations selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/TN, G1049A/C/D/R/S, E453A/D/G/K/Q/V, K111N/R/E, G106A/D/R/SN, G118D, and R88Q. In some embodiments, the patient has mutant PIK3CA containing one or more mutations selected from the group consisting of E542K, E545K, Q546R, H1047L and H1047R. In some embodiments, the patient has breast cancer expressing a PIK3CA mutant selected from the group consisting of H1047D/I/L/N/P/Q/R/T/Y, E545A/D/G/K/L/Q/R/V, E542A/D/G/K/Q/R/V, Q546E/H/K/L/P/R, N345D/H/I/K/S/T/Y, C420R, M1043I/TN, G1049A/C/D/R/S, E453A/D/G/K/Q/V, K111N/R/E, G106A/D/R/SN, G118D, and R88Q. In some embodiments, the patient has breast cancer expressing a PIK3CA mutant selected from the group consisting of H1047R/Y/L, E542K, E545K/D/G/A, Q546K/R/E/L, N345K, C420R, G1049R, R88Q, and M1043I. In some embodiments, the patient has breast cancer expressing a PIK3CA mutant selected from the group consisting of E542K, E545K, Q546R, H1047L and H1047R.

In some embodiments, the patient has mutant PIK3CA containing one mutation selected from the group consisting of E542K, E545K, Q546R, H1047L and H1047R, and a second mutation (e.g., a second mutation selected from E453Q/K, E726K and M1043L/I). In some embodiments, the patient has breast cancer expressing a PIK3CA mutant expressing a double mutation selected from the group consisting of E542K +E453Q/K, E542K +E726K, E542K +M1043L/I; E545K +E453Q/K, E545K +E726K, E545K +M1043L/I; H1047R+E453Q/K, and H1047R +E726K.

PIK3CA-mutant tumor status can be assessed by either central testing of blood or local testing of blood or tumor tissue. In some embodiments, the central test for identification of eligible PIK3CA mutations is the FoundationOne Liquid Clinical Trial Assay performed at Foundation Medicine, Inc. In some embodiments, the local tests of blood or tumor tissue is performed using a Sponsor pre-approved PCR- or NGS-based assay at a CLIA-certified or equivalent laboratory.

The HER2 status of tumors may be assessed by HER2 protein overexpression and/or HER2 gene amplification in tumor specimens using known methods, for example, FDA-approved tests for the detection of HER2 protein overexpression and HER2 gene amplification, preferably performed using FDA-approved tests specific for breast cancer by laboratories with demonstrated proficiency. HER2+ is defined as (i) a HER2 IHC score of 3+; (ii) a HER2 IHC score of 2+ accompanied by a dual-probe ISH HER2/CEP17 ratio of ≥2.0 (preferred); (iii) a fluorescence, chromogenic, or silver ISH test indicating the presence of HER2 gene amplification; or (iv) HER2+ per local clinical guidelines.

In some embodiments, the patient is a female patient with histologically documented locally advanced or metastatic PIK3CA-mutant HER2+ breast cancer. In some embodiments, the patient has a HER2 IHC score of 3 or above. In some embodiments, the patient has a HER2 IHC score of 2 or above accompanied by a dual-probe ISH HER2/CEP17 ratio of no less than 2.0 (preferred). In some embodiments, the patient has a fluorescence, chromogenic, or silver ISH test indicating the presence of HER2 gene amplification;. In some embodiments, the patient is HER2+ per local clinical guidelines. In some embodiments, the patient has a fasting glucose level at or below 140 mg/dL. In some embodiments, the patient has a glycosylated hemoglobin (HbA_{1c}) level below 7%. In some embodiments, the patient has left ventricular ejection fraction (LVEF)≥50%. LVEF can be determined by either echocardiography (ECHO) (preferred) or multiple-gated acquisition (MUGA) scan. In some embodiments, the patient has received a prior HER2-targeted therapy (e.g., trastuzumab and/or pertuzumab) and not have discontinued the prior HER2-targeted therapy (e.g., trastuzumab and/or pertuzumab) because of a toxicity assessed as related to the prior HER2-targeted therapy (e.g., trastuzumab and/or pertuzumab).

Patients may have HR+ or HR− breast cancer. HR+ is defined as expression of ER in ≥1% of cells, or HR+ by local clinical guidelines. HR− is defined as expression of ER in <1% of cells, or HR− by local clinical guidelines. Patients with HR+ breast cancer may be treated with endocrine therapy (e.g., letrozole or fulvestrant), and pre- or perimenopausal patients may also be treated with LHRH agonist therapy, at the investigator's discretion.

In some embodiments, the patient has HR+, locally advanced or metastatic PIK3CA-mutant HER2+breast cancer, and the method of treatment comprises administering to the patient a combination therapy comprising: inavolisib, trastuzumab, pertuzumab, and an endocrine therapy (e.g., letrozole or fulvestrant).

In some embodiments, provided is a method for treating HR+, locally advanced or metastatic PIK3CA-mutant HER2+ breast cancer in a patient, the method comprising administering to the patient a therapeutically effective amount of inavolisib, or a pharmaceutically acceptable salt thereof, trastuzumab, pertuzumab, and an endocrine therapy (e.g., letrozole or fulvestrant). In some embodiments, the endocrine therapy is letrozole administered in accordance with its approved label, for example, administered daily at a dose of 2.5 mg in an oral tablet). In some embodiments, the endocrine therapy is fulvestrant administered in accordance with its approved label, for example, administered once approximately every four weeks at a dose of 500 mg by intramuscular (IM) infusion.

In one embodiment of the methods described herein, a patient has been treated with one or more cancer therapies before administration of a combination therapy described herein. In one embodiment of the methods described herein, the prior therapy comprises a HER2-targeted therapy (e.g., trastuzumab, pertuzumab, or a combination of trastuzumab and pertuzumab). In another embodiment, a patient described herein has not been prior treated with a HER2-targeted therapy (e.g., trastuzumab, pertuzumab, or a combination of trastuzumab and pertuzumab).

In one embodiment of the methods described herein, a patient has breast cancer described herein that is resistant to one or more cancer therapies (e.g., a HER2-targeted therapy such as trastuzumab or pertuzumab). In one embodiment of the methods described herein, resistance to cancer therapy includes recurrence of cancer or refractory cancer. Recurrence may refer to the reappearance of cancer, in the original site or a new site, after treatment. In one embodiment of the methods described herein, resistance to a cancer therapy includes progression of the cancer during treatment with the anti-cancer therapy. In some embodiments of the methods described herein, resistance to a cancer therapy includes cancer that does not response to treatment. The cancer may be resistant at the beginning of treatment or it may become resistant during treatment. In some embodiments of the methods described herein, the cancer is at early stage or at late stage.

Co-administration of inavolisib with a HER2-targeted therapy (e.g., trastuzumab, pertuzumab, or a combination of trastuzumab and pertuzumab) may prevent or delay development of resistance of a tumor (e.g., breast cancer) to a HER2-targeted therapy (e.g., trastuzumab, pertuzumab, or a combination of trastuzumab and pertuzumab). Thus provided is a method of preventing or delaying development of resistance of a tumor (e.g., breast cancer) to a therapy containing a HER2-targeted therapy (e.g., trastuzumab, pertuzumab, or a combination of trastuzumab and pertuzumab), comprising administering a combination therapy detailed herein. In some embodiments, provided is a method of preventing or delaying development of resistance of a tumor (e.g., breast cancer) to a therapy containing trastuzumab and/or pertuzumab, comprising administering a combination therapy comprising trastuzumab and/or pertuzumab, or a combination therapy comprising inavolisib, trastuzumab and pertuzumab. In some embodiments, the combination therapy is administered according to any methods as detailed herein.

### Biomarkers

Breast cancer is a heterogeneous disease with many distinct subtypes as defined by molecular signatures and a diverse array of mutational profiles. In one embodiment, a patient can be tested for PIK3CA/AKT1/PTEN-alteration status. In one embodiment, a patient described herein can be tested for one or more of a phosphatase and tensin homolog (PTEN) mutation, loss of PTEN expression, a phosphatidylinositol-4,5-bisphosphate 3-kinase catalytic subunit alpha (PIK3CA) mutation, a protein kinase B alpha (AKT1) mutation, or a combination thereof. In one embodiment, the loss of PTEN expression is hemizygous or homozygous. In another embodiment, samples of patients described herein can be assessed for additional biomarkers in an effort to identify factors that may correlate with the safety and efficacy of the study treatments.

In one embodiment of the methods described herein, NGS, whole genome sequencing (WGS), other methods, or a combination thereof can be used for DNA obtained from blood samples and tumor tissue from patients described herein. Such samples may be analyzed to identify germline (e.g., BRCA1/2) and somatic alterations that are predictive of response to study drug, are associated with progression to a more severe disease state, are associated with acquired resistance to study drug, or can increase the knowledge and understanding of disease biology. In another embodiment of the methods described herein, patients described herein can have cancer characterized by activation of PI3K/Akt signaling such as activating mutations in PIK3CA or AKT1 as well as through alterations in PTEN, such as those provided herein. In another embodiment, PIK3CA/AKT1/PTEN-altered tumor status will be determined using an NGS assay (e.g., Foundation Medicine, Inc. [FMI]). Review of PIK3CA/AKT1/PTEN-altered status in archival tissue and response measures can be performed on an ongoing basis. Expression of biomarkers (e.g. PTEN) as provided herein can be measured using techniques known in the art such as, for example, immunohistochemistry (IHC).

Circulating tumor DNA (ctDNA) can be detected in the blood of cancer patients with epithelial cancers and may have diagnostic and therapeutic significance (Schwarzenbach et al. Nat Rev Cancer 2011; 11:426-437). For example, the mutational status of tumor cells may be obtained through the isolation of ctDNA (Maheswaran S, et al. N Engl J Med 2008; 359:366-77), and ctDNA has been used to monitor treatment effectiveness in melanoma (Shinozaki M, et al. Clin Cancer Res 2007; 13:2068-74). Blood samples from patients described herein can be collected at screening, at time of first tumor assessment, and/or at the study completion/early termination visit. In one embodiment, the samples are used to evaluate oncogenic genetic alterations at baseline and to assess for the possible emergence of new alteration after treatment with inavolisib and a HER2-targeted therapy.

### EXAMPLES

### Example 1 Inhibition of Breast Cancer Cell Proliferation

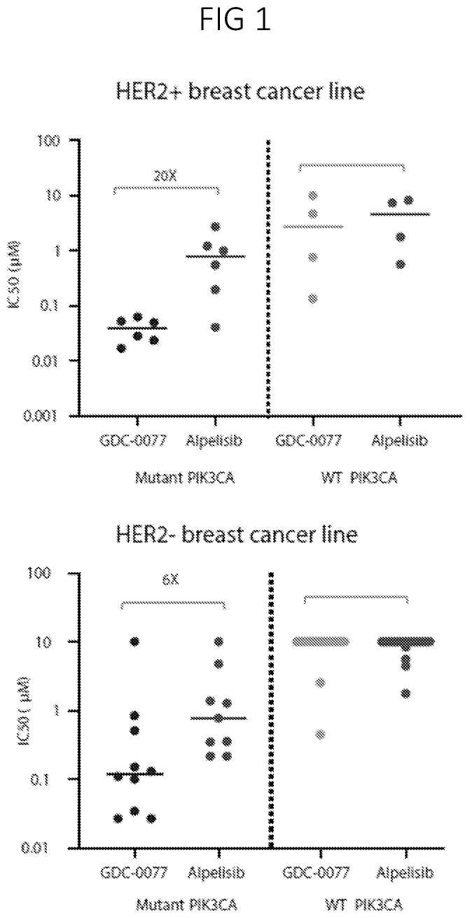

In a cell proliferation assay, we investigated the effect of inavolisib and alpelisib (both PI3Kα specific inhibitors) on vialibility of breast cancer cell lines having different HER2 expression and PIK3CA mutant status. Unexpectedly, we observed a significant difference between the sensitivity of inavolisib and alpelisib in HER2 amplified (˜20 fold difference between the mean IC50 values) versus HER2 negative cell lines (˜6-fold difference between two inhibitors). Both inhibitors were not differentiated in wild-type (WT) cell lines regardless of HER2 status (FIG. 1).

Cell lines were obtained from the ATCC. All cell lines underwent authentication by Short Tandem Repeat profiling, SNP fingerprinting, and mycoplasma testing (Yu M. et al. “A resource for cell line authentication, annotation and quality control” Nature 520, 307-311 (2015)). Cell lines were grown under standard tissue-culture conditions in RPMI media with 10% fetal bovine serum (Gibco, 10082-147), 100 U/mL penicillin-streptomycin (Gibco, 15140-122), 2 mmol/L L-glutamine (Gibco, 15030-081). Cells were treated with compounds for the indicated periods of time.

Viability assay: Cells were seeded (1000-2000 cells/well) in 384-well plates for 16 hours. On day two, nine serial 1:3 compound dilutions were made in DMSO in a 96 well plate. The compounds were then further diluted into growth media using a Rapidplate robot (Zymark Corp., Hopkinton, MA). The diluted compounds were then added to quadruplicate wells in the 384-well cell plate and incubated at 37° C. and 5% CO₂. After 4 days, relative numbers of viable cells were measured by luminescence using CellTiter-Glo® (Promega) according to the manufacturer's instructions and read on a Wallac Multilabel Reader (PerkinElmer, Foster City). The IC₅₀ calculations were carried out using Prism 6.0 software (GraphPad, San Diego). The growth rate (GR) calculations and figures were performed using R scripts based on Hafner, M. et al. “Growth rate inhibition metrics correct for confounders in measuring sensitivity to cancer drugs” Nat. Methods 13, 521-527 (2016).

### Example 2 In Vivo Tumor Growth Inhibition

The in vivo efficacy of the test compounds and combinations was tested in a KPL-4 (HER2-positive, PIK3CA H1047R) breast cancer xenograft model. HCC1954 tumor cells (5×106) were inoculated in the ⅔ mammary fat pads of female NCR nude mice (Taconic Farms, Hudson NY). Tumor volumes were measured using Ultra Cal-IV calipers (Model 54-10-111; Fred V. Fowler Co.; Newton, MA). The following formula was used in Excel, version 11.2 to calculate tumor volume: Tumor Volume (mm³)=(Length×Width²)×0.5. Mice for efficacy studies were distributed into 8-10 mice/group with a mean tumor volume of 200 to 250 mm³ at the initiation of dosing. A linear mixed effect (LME) modeling approach was used to analyze the repeated measurement of tumor volumes from the same animals over time (Pinheiro et al., “Linear and Nonlinear Mixed Effects Models” R package version 3; 1-131 (2017)). Cubic regression splines were used to fit a non-linear profile to the time courses of log² tumor volume at each dose level. These non-linear profiles were then related to dose within the mixed model. Tumor growth inhibition as a percentage of vehicle control (%TGI) was calculated as the percentage of the area under the fitted curve (AUC) for the respective dose group per day in relation to the vehicle, using the following formula: %TGI=100×(1−AUCdose/AUCvehicle).

Inavolisib was formulated in Methylcellulose Tween (MCT) vehicle consisting of 0.5% (w/v) methylcellulose, 0.2% (w/v) polysorbate 80 (Tween-80) and dosed orally by gavage daily at a dose of 25 mg/kg for a period of 28 days. Trastuzumab and pertuzumab were formulated in HB #08 vihicle consisting of HisAc 20 mM, Sucr 240 mM, and TW-20 0.02% (pH 5.5) and dosed by intraperitoneal (i.p.) injection weekly at a dose of 3 mg/kg trastuzumab and 2.5 mg/kg pertuzumab respectively over a period of 4 weeks.

The mice were dosed in four groups (9 mice per group): Group 1, vehicle (MCT); Group 2, trastuzumab+pertuzumab; Group 3, inavolisib; and Group 4, trastuzumab+pertuzumab+inavolisib. Tumor sizes and mouse body weights were recorded twice weekly, and mice with tumor volume exceeding 2000 mm³ or body weight loss of 20% of starting weight were promptly euthanized.

The results are shown in the Table below.

| | | | | Growth Effect | Growth Rate |
|---|---|---|---|---|---|
| | | Growth Contrast | Legacy % TGI | Duration | Contrast |
| Group | Test Article | (Lower, Upper) | (Lower, Upper) | (End Day, Start Day) | (Lower, Upper) |
| 1 | — | 0.000 | 0 | 0.0 | 0.000 |
| | | (0.000, 0.000) | (0, 0) | (31.0, 31.0) | (0.000, 0.000) |
| 2 | trastuzumab + | −0.118 | 134 | 28.3 | −0.095 |
| | pertuzumab | (−0.156, −0.080) | (116, 172) | (28.3, 0.0) | (−0.131, −0.059) |
| 3 | inavolisib | −0.086 | 122 | >31.0 | −0.069 |
| | | (−0.124, −0.048) | (107, 152) | (31.0, 0.0) | (−0.105, −0.033) |
| 4 | trastuzumab + | −0.167 | 148 | >31.0 | −0.126 |
| | pertuzumab + | (−0.205, −0.128) | (125, 203) | (31.0, 0.0) | (−0.162, −0.090) |
| | inavolisib | | | | |

The combination of HER2 inhibitors, trastuzumab and pertuzumab, in combination with inavolisib in a HER2-positive mutant p110α KPL-4 xenograft model showed better response compared with trastuzumab+pertuzumab or inavolisib alone.

### Example 3 Clinical Studies with Inavolisib/Trastuzumab/Pertuzumab Combination

An open-label, Phase I/Ib study of oral daily oral daily inavolisib in combination with HER2 targeted therapies is underway.

Approximately 20 patients may be enrolled in a dose-cohort expansion (Stage II, Arm G) to assess the safety, tolerability, pharmacokinetics, and preliminary anti-tumor activity of inavolisib in combination with trastuzumab and pertuzumab in patients with locally advanced or metastatic PIK3CA-mutant HER2+ breast cancer.

Inavolisib (GDC-0077) is supplied as a tablet in 3 mg or 9 mg strengths. Trastuzumab (Herceptin®) is supplied as a freeze-dried preparation at a nominal content of 440 mg per vial. Pertuzumab is supplied as a single-use formulation containing 30 mg/mL pertuzumab.

### Study Design

Patients with locally advanced or metastatic PIK3CA-mutant HER2+ breast cancer are treated with GDC-0077 (at or below the GDC-0077 MTD or MAD determined in Stage I, Arm A) in combination with trastuzumab and pertuzumab to obtain additional safety, tolerability, and PK data, and preliminary evidence of clinical activity. Patients whose disease is also HR+ may be treated with letrozole or fulvestrant, at the investigator's discretion and according to local guidelines, in addition to GDC-0077, trastuzumab, and pertuzumab.

Inavolisib is administered in combination with trastuzumab and pertuzumab (G+H+P) in 21-day cycles. Patients receive inavolisib at 3, 6, or 9 mg dose on Days 1-21. Patients receive trastuzumab by IV infusion on Day 1 of each 21-day cycle, at a loading dose of 8 mg/kg for Cycle 1 and a dose of 6 mg/kg for subsequent cycles, and pertuzumab by IV infusion on Day 1 of each 21-day cycle, at a loading dose of 840 mg for Cycle 1 and a dose of 420 mg for subsequent cycles, until disease progression or unacceptable toxicity. Patients who received trastuzumab and pertuzumab within 6 weeks of initiating study treatment do not need a loading dose of trastuzumab and pertuzumab on Day 1 of Cycle 1 and receive trastuzumab at 6 mg/kg and pertuzumab at 420 mg on Day 1 of Cycle 1 and subsequent cycles.

Baseline body weight is used to calculate required doses of trastuzumab. The dose of trastuzumab will be re-calculated if a patient's body weight changes from baseline by ≥10%. If the dose is re-calculated because of a ≥10% change in weight from baseline, this weight will then be used as the new baseline to calculate the trastuzumab dose in subsequent cycles.

Administration may be delayed to assess or treat adverse events. If a patient misses a dose of trastuzumab or pertuzumab for one cycle (i.e., two sequential administrations are ≥6 weeks apart), a re-loading dose of 8 mg/kg of trastuzumab or 840 mg of pertuzumab is given. Subsequent doses of 6 mg/kg of trastuzumab and 420 mg of pertuzumab are then given every 21 days.

On study visit days, GDC-0077 is administered first followed by pertuzumab and then trastuzumab. Pertuzumab and trastuzumab are administered by staff trained to monitor for and respond to medical emergencies in a setting with emergency equipment. The initial IV infusion of pertuzumab is administered over 60 (±10) minutes, followed by an observation period of 60 minutes. If the initial infusion is well tolerated, subsequent infusions may be administered over 30 (±10) minutes, followed by an observation period of 30 minutes. The observation period should be completed prior to the subsequent trastuzumab infusion. The initial IV infusion of trastuzumab is administered over 90 (±10) minutes, followed by an observation period of 30 minutes. If the initial infusion is well tolerated, subsequent infusions may be administered over 30 (±10) minutes, followed by an observation period of 30 minutes. Patients can be observed for a longer period at the discretion of the investigator or, if applicable, as per local requirements. Premedication with antipyretics, antihistamines, or corticosteroids may be administered before infusions of pertuzumab and trastuzumab.

Inclusion criteria:
Female participants with locally advanced or metastatic PIK3CA-mutant HER2+ breast cancer;
Left ventricular ejection fraction 50% or greater.

Key eligibility criteria: PIK3CA-mutation in tumor tissue or ctDNA; HER2+ defined as a HER2 IHC score of 3+, a HER2 IHC score of 2+ accompanied by a dual-probe ISH HER2/CEP17 ratio of ≥2.0 (preferred), or a fluorescence, chromogenic, or silver ISH test indicating the presence of HER2 gene amplification, or HER2+ per local clinical guidelines; fasting glucose≤140 mg/dL, HbA1c<7%; and Left ventricular ejection fraction (LVEF)≥50%, as determined by either echocardiography (ECHO) (preferred) or multiple-gated acquisition (MUGA) scan, at screening. Patients who have received prior HER2-directed therapies, including trastuzumab and/or pertuzumab, must not have discontinued prior trastuzumab and/or pertuzumab because of a toxicity assessed as related to one or both agents.

Patients with HR+ breast cancer may be treated with endocrine therapy (i.e., letrozole or fulvestrant), and pre- or perimenopausal patients may also be treated with LHRH agonist therapy, at the investigator's discretion.

Exclusion Criteria:
Current uncontrolled hypertension (systolic blood pressure>150 mmHg and/or diastolic blood pressure>100 mmHg) or unstable angina;
History of congestive heart failure (CHF) of New York Heart Association (NYHA) classification Class II or higher, or serious cardiac arrhythmia requiring treatment (excluding atrial fibrillation or paroxysmal supraventricular tachycardia);
History of myocardial infarction within 6 months prior to initiation of study treatment;
Prior ejection fraction decrease on trastuzumab (History of LVEF decline to below 40% during or after prior treatment with trastuzumab);
History of exposure to cumulative dose of doxorubicin (or equivalent anthracycline exposure) >360 mg/m² of body surface area or its equivalent;
Symptomatic active lung disease, including pneumonitis or interstitial lung disease;

History of prior significant toxicity related to trastuzumab and/or pertuzumab requiring discontinuation of treatment.

Anti-tumor activity is assessed at screening and every 8 weeks by RECIST v1.1. Clinical benefit rate (CBR) defined as complete or partial response, or stable disease lasting≥24 weeks.

Pharmacodynamic activity is assessed pre- and on-study (after 2 weeks of daily inavolisib treatment), in tumor biopsies via immunohistochemistry (IHC), and in ctDNA samples (by Foundation ACT).

The important safety risks for trastuzumab and pertuzumab, for example, embryo-fetal toxicity, hypersensitivity/anaphylaxis and infusion-related reactions, decrease in LVEF, diarrhea, rash/skin reactions, stomatitis/oral mucositis, and pulmonary toxicity, are managed in accordance with the Investigator's Brochures for Trastuzumab and Pertuzumab.

### Example 4 Clinical Studies with Inavolisib and Paclitaxel with or without Targeted Therapies

A Phase Ib, open-label, dose-escalation and dose-expansion study evaluating the safety, tolerability, pharmacokinetics, and preliminary antitumor activity of inavolisib in combination with paclitaxel and with or without targeted therapies in patients with locally advanced or metastatic solid tumors is underway.

The aim is to evaluate the safety, pharmacokinetics, pharmacodynamic (PD) effects, and preliminary anti-tumor activity of inavolisib administered in combination with paclitaxel in patients with locally advanced or metastatic solid tumors, and of inavolisib administered in combination with paclitaxel, trastuzumab and pertuzumab in patients with locally advanced or metastatic PIK3CA-mutated, HER2-positive breast cancer.

### Interventions

The study is an open-label design and the treatments include: Inavolisib—6 mg/9 mg taken orally every day and potentially on an intermittent weekly schedule of 6/1 or 5/2, until disease progression or unacceptable toxicity. Paclitaxel—Intravenous infusion 80 mg/m² dose weekly, until disease progression or unacceptable toxicity. Trastuzumab—Intravenous infusion every 3 weeks. Loading dose of 8 mg/kg for Cycle 1 and a dose of 6 mg/kg for subsequent cycles, until disease progression or unacceptable toxicity. Pertuzumab—Intravenous infusion every 3 weeks. Loading dose of 840 mg for the first cycle and a dose of 420 mg for subsequent cycles, until disease progression or unacceptable toxicity.

Up to 120 patients at various hospital locations around the world will take part in this study. The study is divided into two parts. Part 1, Arm A is the dose-escalation (dose-finding) part of the study. Part 2 consists of Arm A and Arm B, which will be treatment expansions after dose-finding is complete. Participants will be allocated to the different treatments based on inclusion and exclusion criteria for Part 1 and Part 2:
Part 1, Arm A: inavolisib will be tested at different doses and schedules in combination with paclitaxel in up to 24 patients with locally advanced or metastatic cancer.
Part 2, Arm A: a dose and schedule of inavolisib determined to be safe in Part 1, Arm A will be tested in combination with paclitaxel, in approximately 76 patients with locally advanced or metastatic PIK3CA mutated (altered gene) cancer.
Part 2, Arm B: a dose and schedule of inavolisib determined to be safe in Part 1, Arm A will be tested in combination with paclitaxel, trastuzumab and pertuzumab in approximately 20 patients with locally advanced or metastatic PIK3CA-mutated (altered gene) HER2 positive breast cancer

Patients will have the following assessments and measurements:
1. Vital signs—temperature, pulse rate, blood pressure, breathing rate and oxygen level;
2. Complete or limited physical exam;
3. Assessment of performance status (Eastern Cooperative Oncology Group daily functioning);
4. Electrocardiogram (ECG);
5. Urine sample for standard laboratory tests;
6. Blood samples for standard laboratory tests and to measure pharmacokinetics;
7. Tumor tissue biopsies;
8. Tumor assessments: scans of internal organs and bones that may include: computed tomography (CT) scan, magnetic resonance imaging (MRI) scan, and bone scan;
9. Eye examination.

### Primary Outcome Measure

Safety Objectives:
1. Incidence and nature of dose-limiting toxicities (Part 1 only) measured using adverse events (graded by NCI CTCAE v5.0) recorded within the first 28 days (cycle 1) of study treatment;
2. Incidence, type, and severity of adverse events including serious adverse events graded by NCI CTCAE v5.0 recorded throughout the study;
3. Targeted vital signs measured using standard techniques assessed by site staff at baseline and weekly/monthly during study treatment;
4. Targeted clinical laboratory test results measured using standard hospital laboratory analyses at baseline and taken every week/cycle, including ECGs, recorded by taken by site staff at baseline and every cycle during study treatment.

### Secondary Outcome Measures

Pharmacokinetic Objective (Secondary Objective):

Plasma concentration of inavolisib administered in combination with paclitaxel (Arm A), or with paclitaxel, trastuzumab, and pertuzumab (Arm B) measured by a bioanalytical laboratory at Cycles 1-3. The following PK parameters will be determined as appropriate:
1. Area under the concentration-time curve (AUC)
2. Maximum plasma concentration (Cmax)
3. Minimum plasma concentration (Cmin)
4. Additional plasma PK parameters as warranted

Activity/Efficacy Objectives (Secondary Objectives):

To make a preliminary assessment of the anti-tumor activity of:
1. Inavolisib administered in combination with paclitaxel in patients with locally advanced or metastatic PIK3CA-mutated solid tumors (*Part 2 Arm A);
2. Inavolisib administered in combination with paclitaxel, trastuzumab and pertuzumab in patients with locally advanced or metastatic PIK3CA mutated, HER2-positive breast cancer (#Part 2 Arm B).

The corresponding endpoints are as follows:
1. Objective response rate (ORR) defined as a complete recovery (CR) or partial recovery (PR) on two consecutive occasions weeks ≥4 weeks apart, as determined by the investigator through use of RECIST v1.1 every 8(*) or 9(#) weeks during study treatment;
2. Best overall response (BOR) defined as the proportion of patients with a CR or PR, as determined by the investigator through use of RECIST v1.1 every 8(*) or 9(#) weeks during study treatment;
3. Duration of response (DOR) defined as the time from the first occurrence of a documented objective response to disease progression, as determined by the investigator through use of RECIST v1.1, or death, whichever occurs first, every 8(*) or 9(#) weeks during study treatment;
4. Clinical benefit rate (CBR) defined as the percentage of patients achieving confirmed RECIST v1.1 defined CR, PR, or stable disease (SD; non-complete response/non-progressive disease for patients with non-measurable disease at baseline) ≥24 weeks, as determined by the investigator through use of RECIST v1.1 every 8(*) or 9(#) weeks during study treatment;
5. Progression-free survival (PFS) defined as the time from the first study treatment (Day 1) to the first occurrence of disease progression, as determined by the investigator through use of RECIST v1.1 every 8(*) or 9(#) weeks during study treatment, or death, whichever occurs first.

Participant Inclusion Criteria:
1. Signed Informed Consent Form;
2. Aged over 18 years and over;
3. Evaluable or measurable disease per RECIST, v1.1;
4. Eastern Cooperative Oncology Group (ECOG) performance status of 0 or 1;
5. Life expectancy of 12 weeks;
6. Adequate hematologic and organ function within 14 days prior to initiation of study treatment, defined by the following:
   6.1. Absolute neutrophil count 1500/l,
   6.2. Hemoglobin 9 g/dl,
   6.3. Platelet count 100,000/l,
   6.4. Fasting glucose 126 mg/dL or 7 mmol/l and glycosylated hemoglobin (HbA1C) 5.7%,
   6.5. Total bilirubin 1.5 upper limit of normal (ULN),
   6.6. Serum albumin 2.5 g/dl or 25 g/l,
   6.7. AST and ALT 2.5 ULN with the following exception: patients with documented liver metastases may have AST and ALT 5.0 ULN,
   6.8. Serum creatinine 1.5 ULN or creatinine clearance 50 ml/min on the basis of the Cockcroft-Gault glomerular filtration rate estimation;
7. Consent to provide fresh (preferred) or archival tumor tissue specimen;
8. For women of childbearing potential: agreement to remain abstinent (refrain from heterosexual intercourse) or use a highly effective form of contraceptive method with a failure rate of 1% per year in combination with use of male condom with spermicide (for male partners), unless male sterilization has been confirmed;
9. For men: agreement to remain abstinent (refrain from heterosexual intercourse) or use highly effective contraceptive measures, and agreement to refrain from donating sperm.

Inclusion Criteria Specific to Patients Enrolling in Part 1, Arm A: Histologically documented, locally advanced, recurrent, or metastatic, incurable solid tumor malignancy that has progressed after available standard systemic therapies; or for whom standard therapy has proven to be ineffective or intolerable; or for whom a clinical trial of an investigational agent is a recognized standard of care. If there are other available SOC therapies, these will be discussed with the patient and documented before informed consent is obtained.

Inclusion Criteria Specific to Patients Enrolling in Part 2, Arm A Expansion Cohorts:
1. Histologically documented, locally advanced, recurrent, or metastatic, incurable solid tumor malignancy with a known PIK3CA mutation that has progressed after at least one available standard systemic therapy in the metastatic setting;
2. Confirmation of biomarker eligibility: valid results from central testing of blood or local testing of blood or tumor tissue documenting PIK3CA-mutated tumor status is required for patients enrolling to Part 2, Arm A, expansion cohorts.

Inclusion Criteria Specific to Patients Enrolling in Part 2, Arm B:
1. Patients with histologically documented locally advanced or metastatic PIK3CA-mutated HER2-positive breast cancer;
2. Patients may present with either: de novo metastatic HER2-positive disease for which they have not received any systemic HER2-positive anti-cancer treatment recurrent locally advanced or metastatic disease following prior HER2-positive targeted treatment for early breast cancer, where the diagnosis has been based on the biopsy of the locally recurrent or metastatic disease and the patient has progressed following (neo)adjuvant HER2-positive targeted therapy with a treatment-free interval of 6 months;
3. Documented HER2-positive and either HR-positive or HR-negative breast cancer according to ASCO/CAP guidelines based on local assessment HER2-positive;
4. Confirmation of biomarker eligibility: valid results from central testing of blood documenting PIK3CA-mutated tumor status is required for patients enrolling to Part 2, Arm B;
5. Left ventricular ejection fraction (LVEF) 50%, as determined by either echocardiography (ECHO) (preferred) or multiple gated acquisition (MUGA) scan, at screening;
6. For women of childbearing potential: agreement to remain abstinent (refrain from heterosexual intercourse) or use a highly effective form of contraceptive method with a failure rate of 1% per year, and agreement to refrain from donating eggs, during the treatment period and for at least 60 days after the last dose of inavolisib, at least 6 months after the last dose of paclitaxel, at least 7 months after the last dose of pertuzumab and the last dose of trastuzumab.

### Participant Exclusion Criteria

1. Metaplastic breast cancer;
2. Any history of leptomeningeal disease;
3. Type 2 diabetes requiring ongoing systemic treatment at the time of study entry; or any history of Type 1 diabetes;
4. Inability or unwillingness to swallow pills;
5. Malabsorption syndrome or other condition that would interfere with enteral absorption;
6. Known and untreated, or active CNS metastases (progressing or requiring anticonvulsants or corticosteroids for symptomatic control);
7. Uncontrolled pleural effusion or ascites requiring recurrent drainage procedures twice per month or more frequently;
8. Any active infection that, in the opinion of the investigator, could impact patient safety; or, serious infection requiring IV antibiotics within 7 days prior to Day 1 of Cycle 1;
9. Any concurrent ocular or intraocular condition (e.g., cataract or diabetic retinopathy) that, in the opinion of the investigator or study ophthalmologist, would require medical or surgical intervention during the study period to prevent or treat vision loss that might result from that condition;
10. Active inflammatory (e.g., uveitis or vitritis) or infectious (e.g., conjunctivitis, keratitis, scleritis, or endophthalmitis) conditions in either eye or history of idiopathic or autoimmuneassociated uveitis in either eye;
11. Patients requiring any daily supplemental oxygen;
12. History of or active inflammatory disease (e.g., Crohn's disease or ulcerative colitis), or any active bowel inflammation (including diverticulitis);
13. Patients currently receiving immunosuppressants (e.g., sulfasalazines) are considered to have active disease; therefore, they are ineligible;
14. Symptomatic hypercalcemia requiring continued use of bisphosphonate or denosumab therapy;
15. Clinically significant history of liver disease, including severe liver impairment (Child-Pugh Class B/C), viral or other hepatitis, current alcohol abuse, or cirrhosis;
16. Known HIV infection;
17. Any other diseases, active or uncontrolled pulmonary dysfunction, metabolic dysfunction, physical examination finding, or clinical laboratory finding giving reasonable suspicion of a disease or condition that contraindicates the use of an investigational drug, that may affect the interpretation of the results, or renders the patients at high risk from treatment complications;
18. Significant traumatic injury or major surgical procedure within 4 weeks prior to initiation of study treatment;
19. Radiation therapy (other than palliative radiation to bony metastases) as cancer therapy within 4 weeks prior to initiation of study treatment;
20. Palliative radiation to bony metastases within 2 weeks prior to initiation of study treatment;
21. Unresolved toxicity from prior therapy, except for the following: Alopecia Grade 1 and peripheral neuropathy;
22. Inability to comply with study and follow-up procedures;
23. History of other malignancy within 5 years prior to screening, with the exception of patients with a negligible risk of metastasis or death and/or treated with expected curative outcome (such as appropriately treated carcinoma in situ of the cervix, non-melanoma skin carcinoma, localized prostate cancer, ductal carcinoma in situ, or Stage I uterine cancer);
24. History of or active ventricular dysrhythmias or congestive heart failure requiring medication or coronary heart disease that is symptomatic;
25. Clinically significant electrolyte abnormalities (e.g., hypokalemia, hypomagnesemia, hypocalcemia);
26. Congenital long QT syndrome or QT interval corrected with Fridericia's formula (QTcF) 470 ms demonstrated by at least two ECGs 30 minutes apart, or family history of sudden unexplained death or long QT syndrome;
27. Current treatment with medications that are well known to prolong the QT interval;
28. Allergy or hypersensitivity to components of the inavolisib formulation and paclitaxel;
29. Pregnancy, lactation, or intention to become pregnant or fathering a child during the study;
30. Women of childbearing potential (including those who have had a tubal ligation) must have a negative serum pregnancy test result within 14 days prior to initiation of study treatment.

Exclusion Criteria Specific to Patients Enrolling Part 1, Arm A:
1. History of prior significant toxicity related to a PI3K, AKT, or mTOR inhibitor requiring discontinuation of treatment. Patients may have received prior treatment with a PI3K, AKT, or mTOR inhibitor;
2. History of prior significant toxicity related to paclitaxel treatment requiring discontinuation of treatment. Patients may have received prior treatment with paclitaxel;
3. Treatment with chemotherapy, immunotherapy, or biologic therapy as anti-cancer therapy within 21 days prior to initiation of study treatment, except for the following: Kinase inhibitors, approved by regulatory authorities, may be used up to 2 weeks prior to initiation of study treatment, provided any drug-related toxicity has resolved up to Grade 1 and prior approval is obtained from the Medical Monitor. Treatment with an investigational agent within 3 weeks or five half-lives prior to initiation of study treatment, whichever is shorter. A shorter washout period may be allowed if the patient has adequately recovered from any clinically relevant toxicity and with prior approval from the Medical Monitor.
4. Prior anti-cancer therapy that fulfills the following criteria: High dose chemotherapy requiring stem-cell support; Irradiation to 25% of bone marrow-bearing areas.

Exclusion Criteria Specific to Patients Enrolling Part 2, Arm A:
1. History of prior significant toxicity related to a PI3K, AKT, or mTOR inhibitor requiring discontinuation of treatment;
2. Prior treatment with any PI3K-specific inhibitor;
3. History of prior significant toxicity related to paclitaxel treatment requiring discontinuation of treatment. Patients may have received prior treatment with paclitaxel;
4. Treatment with chemotherapy, immunotherapy, or biologic therapy as anti-cancer therapy within 21 days prior to initiation of study treatment, except for the following: Kinase inhibitors, approved by regulatory authorities, may be used up to 2 weeks prior to initiation of study treatment, provided any drug-related toxicity has resolved up to Grade 1 and prior approval is obtained from the Medical Monitor Treatment with an investigational agent within 3 weeks or five half-lives prior to initiation of study treatment, whichever is shorter;
5. Prior anti-cancer therapy that fulfills the following criteria: high dose chemotherapy requiring stem-cell support Irradiation to 25% of bone marrow-bearing areas.

Exclusion Criteria Specific to Patients Enrolling Part 2, Arm B:
1. Prior treatment with any PI3K, AKT, or mTOR inhibitor, or any agent whose mechanism of action is to inhibit the PI3K/AKT/mTOR pathway;
2. Any prior systemic anti-cancer therapy for locally advanced or metastatic HER2-positive breast cancer prior to initiation of study treatment;
3. Current uncontrolled hypertension (systolic blood pressure over 150 mmHg or diastolic blood pressure over 100 mmHg) or unstable angina;
4. History of congestive heart failure (CHF) of New York Heart Association (NYHA) classification Class II or higher, or serious cardiac arrhythmia requiring treatment (excluding atrial fibrillation or paroxysmal supraventricular tachycardia);
5. History of myocardial infarction within 6 months prior to initiation of study treatment;
6. History of LVEF decline to below 40% during or after prior treatment with trastuzumab;
7. History of exposure to cumulative dose of doxorubicin (or equivalent anthracycline exposure) 360 mg/m² of body surface area or its equivalent;
8. Symptomatic active lung disease, including pneumonitis or interstitial lung disease;
9. History of prior significant toxicity related to paclitaxel, trastuzumab or pertuzumab requiring discontinuation of treatment.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method of treating HER2-positive breast cancer in a patient having locally advanced or metastatic PIK3CA-mutated HER2-positive breast cancer, the method comprising administering to the patient a combination therapy comprising:
inavolisib;
(ii) trastuzumab;
(iii) pertuzumab; and optionally
(iv) paclitaxel,
wherein said combination therapy is administered over a 21-day cycle.

2. A method of treating HER2-positive breast cancer in a patient having locally advanced or metastatic PIK3CA-mutated HER2-positive breast cancer, the method comprising administering to the patient a combination therapy comprising a dosing regimen comprising:
a. administering inavolisib QD on days 1-21 of a first 21-day cycle;
b. administering trastuzumab on day 1 of a first 21-day cycle; and
c. administering pertuzumab on day 1 of a first 21-day cycle.

3. A method of treating HER2-positive breast cancer in a patient having locally advanced or metastatic PIK3CA-mutated HER2-positive breast cancer, the method comprising administering to the patient a combination therapy comprising a dosing regimen comprising:
a. administering paclitaxel on days 1, 8 and 15 of a first 21-day cycle;
b. administering inavolisib QD on days 2-21 of a first 21-day cycle;
c. administering trastuzumab on day 2 of a first 21-day cycle;
d. administering pertuzumab on day 2 of a first 21-day cycle;
and the dosing regimen further comprises up to five additional 21-day cycles comprising:
e. administering paclitaxel on days 1, 8 and 15 of each additional 21-day cycle;
f. administering inavolisib on days 1-21 of each additional 21-day cycle;
g. administering trastuzumab on day 1 of each additional 21-day cycle; and
h. administering pertuzumab on day 1 of each additional 21-day cycle.

4. The method of claim 2, further comprising one or more additional 21-day cycles comprising:
a. administering inavolisib on days 1-21 of each additional 21-day cycle;
b. administering trastuzumab on day 1 of each additional 21-day cycle; and
c. administering pertuzumab on day 1 of each additional 21-day cycle.

5. The method of claim 1, wherein inavolisib is administered at an amount of 9 mg.

6. The method of claim 5, wherein inavolisib is administered at an amount of 9 mg in an oral tablet.

7. The method of claim 4, wherein trastuzumab is administered at a loading dose of 8 mg/kg for the first 21-day cycle and a dose of 6 mg/kg for each additional 21-day cycle by intravenous (IV) infusion.

8. The method of claim 4, wherein pertuzumab is administered at a loading dose of 840 mg for the first 21-day cycle and a dose of 420 mg for each additional 21-day cycle by IV infusion.

9. The method of claim 3, wherein paclitaxel is administered at a weekly dose of 80 mg/m² for the first 21-day cycle and up to five additional 21-day cycles by IV infusion.

10. The method of claim 1, wherein the patient has left ventricular ejection fraction (LVEF) 50% or greater.

11. The method of claim 1, wherein the patient has hormone receptor positive (HR+) locally advanced or metastatic PIK3CA-mutated HER2-positive breast cancer.

12. The method of claim 11, wherein the method further comprising administering to the patient fulvestrant.

13. The method of claim 12, wherein fulvestrant is administered once about every four weeks at a dose of 500 mg by intramuscular (IM) infusion.

14. The method of claim 11, wherein the method further comprising administering to the patient letrozole.

15. The method of claim 14, wherein letrozole is administered daily at a dose of 2.5 mg in an oral tablet.

16. A method of inhibiting tumor growth or producing/increasing tumor regression in a patient having locally advanced or metastatic PIK3CA-mutated HER2-positive breast cancer, the method comprising administering to the patient a combination therapy comprising inavolisib, trastuzumab and pertuzumab according to the method of claim 1.

17. A method of preventing or delaying development of resistance of a breast cancer to a HER2-targeted therapy, said method comprising administering a combination therapy comprising inavolisib, trastuzumab and pertuzumab according to the method of claim 1.

18. The method of claim 4, wherein inavolisib is administered at an amount of 9 mg in an oral tablet.
